(19) United States
(12) Patent Application Publication
(10) Pub. No.: US 2003/0180808 A1
(43) Pub. Date: Sep. 25, 2003

(54) Drug signatures

(76) Inventor: Georges Natsoulis, Kensington, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/378,002

(22) Filed: Feb. 28, 2003

Related U.S. Application Data
(60) Provisional application No. 60/360,728, filed on Feb. 28, 2002.

(51) Int. Cl. G01N 33/53; G01N 33/567; G06F 19/00; G01N 33/48; G01N 33/50; C12Q 1/68

(52) U.S. Cl. 435/7.1; 435/7.2; 702/19; 435/6

(57) ABSTRACT

Methods for deriving and using Group Signatures and Drug Signatures are provided, wherein Group Signatures comprise a plurality of genes, modulated expression of which is characteristic and specific of a group of related drug compounds, and wherein Drug Signatures comprise a plurality of genes, modulated expression of which is characteristic and specific for individual drug compounds.

This application claims the benefit of U.S. Provisional Application No. 60/360,728, filed Feb. 28, 2002.

### FIELD OF THE INVENTION

This invention relates to the fields of genomics, chemistry, and drug discovery. More particularly, the invention relates to methods and systems for grouping and classifying compounds by their activity and genomic effect in vivo, and methods and systems for predicting the activity and side effects of a compound in vivo.

### BACKGROUND OF THE INVENTION

Genomic sequence information is now available for several organisms, and additional data is added continuously. However, only a small fraction of the open reading frames now sequenced correspond to genes of known function: the function of most polynucleotide sequences, and many encoded proteins, is still unknown. These genes are now studied by means of, inter alia, polynucleotide arrays, which quantify the amount of mRNA produced by a test cell (or organism) under specific conditions. “Chemogenomic annotation” is the process of determining the transcriptional and bioassay response of one or more genes to exposure to a particular chemical, and defining and interpreting such genes in terms of the classes of chemicals for which they interact. A comprehensive library of chemogenomic annotations would enable one to design and optimize new pharmaceutical lead compounds based on the probable transcriptional and biomolecular profile of a hypothetical compound with certain characteristics. Additionally, one can use chemogenomic annotations to determine relationships between genes (for example, as members of a signal pathway or protein-protein interaction pair), and aid in determining the causes of side effects and the like. Finally, presenting the drug design researcher with a body of chemogenomic annotation information will generate research hypotheses that will stimulate follow-on experimental design.

Several genomic database models have been disclosed. Sabatini et al., U.S. Pat. No. 5,966,712 disclosed a database and system for storing, comparing and analyzing genomic data. Maslyn et al., U.S. Pat. No. 5,953,727 disclosed a relational database for storing genomic data. Kohler et al., U.S. Pat. No. 5,523,208 disclosed a database and method for comparing polynucleotide sequences and the predicted functions of their encoded proteins. Fujiyama et al., U.S. Pat. No. 5,706,498 disclosed a database and retrieval system, for identifying genes of similar sequence.

Sabry et al., WO00/70528 disclosed methods for analyzing compounds for drug discovery using a cellular informatics database. The system images cells that have been manipulated or exposed to test compounds, converting the resulting data into a database. Sabry further describes constructing a database of “cellular fingerprints” comprising descriptors of cell-compound interactions, where the descriptors are a collection of identified data/phenotype variations that characterize the interaction with compounds of known action, constructing a phylogenetic tree from the descriptors, and determining the statistical significance of each descriptor. The descriptor for a new compound can be compared to the phylogenetic tree to determine its most likely mode of action.

Winslow et al., WO00/65523, disclosed a system comprising a database containing biological information which is used to generate a data structure having at least one associated attribute, a user interface, an equation generation engine operative to generate at least one mathematical equation from at least one hierarchical description, and a computational engine operative on the mathematical equation to model dynamic subcellular and cellular behavior. The system is intended to access and tabulate genetic information contained within proprietary and nonproprietary databases, combine that data with functional information regarding the biochemical and biophysical role of gene products, and based on this information formulate, solve and analyze computational models of genetic, biochemical and biophysical processes within cells.

Gould-Rothberg et al., WO00/63435, disclosed a method for identifying hepatotoxic agents by exposing a test cell population comprising a cell capable of expressing one or more nucleic acids sequences responsive to troglitazone (an anti-diabetes drug discovered to cause liver damage in some patients during phase III trials), contacting the test cell population with the test agent and comparing the expression of the nucleic acids sequences in a reference cell population. An alteration in expression of the nucleic acids sequences in the test cell population compared to the expression of the gene in the reference cell population indicates that the agent is hepatotoxic. Gould-Rothberg et al., WO00/37685, disclosed a method for identifying psychoactive agents that lack motor involvement, by identifying genes transcriptionally activated in rat brain striatum in response to haloperidol. Compounds that do not induce these genes are believed to not result in side effects.

Thorp, WO99/06839, describes a protein database, for screening with combinatorial chemical libraries. The database relates target and reference proteins, compounds and assays. The protein descriptors include molecular weight, activity, hydrophobicity, etc., and also their binding patterns with aptamers. Similarity of a target protein to a reference protein is used to weight the combinatorial libraries examined more toward compounds that bind the most similar reference proteins.

Friend et al., U.S. Pat. No. 6,203,987, discloses a method for comparing array profiles by grouping genes into co-regulated sets (“genesets”). Friend et al. disclose an embodiment in which the expression profile obtained in response to a drug is projected into a geneset, and compared with other genesets to determine the biological pathways affected by the drug. In another embodiment, the projected profiles of drug candidates are compared with the profiles of known drugs to identify possible replacements for existing drugs.

Tamayo et al., EP 1037158, disclosed a method for organizing genomic data using Self Organizing Maps to cluster gene expression data into similar sets. The method can be used to identify drug targets, by identifying which genes move from their expression clusters after a test cell is exposed to a given compound.

Tryon et al., WO01/25473, disclosed a method for constructing expression profiles of genes in response to a drug. In this method, a number of genes are selected on the basis of their expected interaction with the drug or condition to be examined, and their expression in cell culture is measured in response to administration of the drug.

### SUMMARY OF THE INVENTION

One aspect of the invention is a method for creating a Group Signature for a plurality of compounds having related activities, said method comprising: a) providing a plurality of expression datasets, each expression dataset comprising the expression response of a first plurality of genes in a subject cell following exposure to a compound, wherein said plurality of expression datasets comprises an expression dataset for each of a plurality of test compounds having a similar or identical biological activity, and an expression dataset for each of a plurality of control compounds that lack the biological activity of the test compounds; b) deriving a discrimination metric that distinguishes the plurality of test compounds from the control compounds based on gene expression to provide a distinctive gene set; and c) selecting a second plurality of genes from said distinctive gene set to provide a Group Signature for said plurality of test compounds.

Another aspect of the invention is a method for creating a Group Signature for a plurality of compounds having related activities, said method comprising: a) providing a plurality of test compounds having a similar or identical biological activity, and a plurality of control compounds that lack the biological activity of the test compounds; b) contacting each compound with a subject cell; c) measuring the expression response of a first plurality of genes for each subject cell to provide an expression dataset for each compound; d) ordering the expression datasets by Principal Component Analysis to provide a plurality of principal components; e) identifying the Principal Component that distinguishes the plurality of test compounds from the plurality of control compounds to the greatest degree, to provide a test Principal Component; f) identifying the genes that distinguish the test Principal Component from the control compounds to the greatest degree to provide a distinctive gene set; and g) selecting a second plurality of genes from said distinctive gene set to provide a Group Signature for said plurality of test compounds.

Another aspect of the invention is a method for creating a Drug Signature capable of distinguishing the activity of a selected drug compound from a plurality of compounds having related activities, said method comprising: a) providing a plurality of expression datasets, each expression dataset comprising the expression response of a plurality of genes in a subject cell following exposure to a compound, wherein said plurality of expression datasets comprises an expression dataset for said selected drug compound and an expression dataset for each of a plurality of test compounds having a similar or identical biological activity; b) deriving a discrimination metric that distinguishes the selected drug compound from the plurality of test compounds based on gene expression to provide a distinctive gene set; and c) selecting a plurality of genes from said distinctive gene set to provide a Drug Signature for said selected drug compound.

Another aspect of the invention is a method for creating a Drug Signature capable of distinguishing the activity of a selected drug compound from a plurality of compounds having related activities, said method comprising: a) providing said selected drug compound and a plurality of test compounds having a similar or identical primary biological activity; b) contacting each compound with a subject cell; c) measuring the expression response of a first plurality of genes for each subject cell to provide an expression dataset for each compound; d) ordering the expression datasets by Principal Component Analysis to provide a plurality of principal components; e) identifying the Principal Component that distinguishes the selected drug compound from said plurality of test compounds to the greatest degree, to provide a distinguishing Principal Component; f) identifying the genes that contribute to the distinguishing Principal Component to the greatest degree to provide a distinguishing gene set; and g) selecting a second plurality of genes from said distinguishing gene set to provide a Drug Signature for said selected drug compound.

Another aspect of the invention is a Group Signature database comprising: a plurality of Group Signature records, wherein each Group Signature record comprises indicia of at least one compound, wherein all compounds within a Group exhibit a similar or identical primary bioactivity; indicia of a set of genes, wherein the expression of said genes is modulated in response to exposure to a compound having a primary bioactivity similar or identical to the primary bioactivity of a compound indicated in the Group record, and wherein said set of genes distinguishes said Group from all other Groups within said Group Signature database. A further aspect of this invention is a Group Signature database comprising stress records, wherein each stress record comprises: an indicia of a stress; and indicia of a set of genes, wherein expression of said genes is modulated in response to said stress, and wherein said set of genes distinguishes said stress from all other stresses and Groups within said Group Signature database.

Another aspect of the invention is a Drug Signature database comprising: a plurality of Drug Signature records, wherein each Drug Signature record comprises indicia of one compound; and indicia of a set of genes, wherein expression of said genes is modulated in response to exposure to said compound, and wherein said set of genes distinguishes said compound from all other compounds within said Drug Signature database.

Another aspect of the invention is a method for determining the activity of a drug candidate, said method comprising: a) providing a Group Signature database, said Group Signature database comprising a plurality of Group Signature records, wherein each Group Signature record comprises indicia of at least one compound, wherein all compounds within a Group exhibit a similar or identical primary bioactivity; and indicia of a set of genes, wherein expression of said genes is modulated in response to exposure to a compound having a primary bioactivity similar or identical to the primary bioactivity of a compound indicated in the Group record, and wherein said set of genes distinguishes said Group from all other Groups within said Group Signature database; b) providing a drug candidate expression dataset for said drug candidate, said drug candidate expression dataset comprising the expression response of a plurality of genes in a subject cell following exposure to said drug candidate; c) comparing said drug candidate expression dataset with each Group Signature; d) selecting the Group Signature most similar to said drug candidate expression dataset; e) identifying the activity of the drug candidate to be the primary bioactivity exhibited by the compounds within the most similar Group Signature.

Another aspect of the invention is a method for designing a Group Signature reagent, comprising: a) providing a plurality of expression datasets, each expression dataset comprising the expression response of a first plurality of genes in a subject cell following exposure to a compound, wherein said plurality of expression datasets comprises an expression dataset for each of a plurality of test compounds having a similar or identical biological activity, and an expression dataset for each of a plurality of control compounds that lack the biological activity of the test compounds; b) deriving a discrimination metric that distinguishes the plurality of test compounds from the control compounds based on gene expression to provide a distinctive gene set; c) selecting a second plurality of genes from said distinctive gene set to provide a Group Signature for said plurality of test compounds; and d) providing a set of polynucleotide probes capable of hybridizing specifically to one or more sequences of said second plurality of genes in said Group Signature to provide a Group Signature probe set. The invention further includes the probe sets designed by the above methods and kits, containing such probe sets.

Another aspect of the invention is a method for designing a Drug Signature reagent, comprising: a) providing a plurality of expression datasets, each expression dataset comprising the expression response of a plurality of genes in a subject cell following exposure to a compound, wherein said plurality of expression datasets comprises an expression dataset for said selected drug compound and an expression dataset for each of a plurality of test compounds having a similar or identical biological activity; b) deriving a discrimination metric that distinguishes the plurality of test compounds from the control compounds based on gene expression to provide a distinctive gene set; c) selecting a plurality of genes from said distinguishing gene set to provide a Drug Signature for said selected drug compound; and d) providing a set of polynucleotide probes capable of hybridizing specifically to the sequences of said genes in said Drug Signature to form a Drug Signature probe set. The invention further includes the probe sets designed by the above methods and kits, containing such probe sets.

Another aspect of the invention is a method for determining the activity of a drug candidate, said method comprising: a) providing a Group Signature Array, said Group Signature Array comprising a solid support having affixed thereto a plurality of Group Signature probe sets, wherein each Group Signature probe set comprises a set of polynucleotide probes capable of hybridizing specifically to the sequences of the genes in each Group Signature, wherein said Group Signatures are obtained by: i) providing a plurality of expression datasets, each expression dataset comprising the expression response of a plurality of genes in a subject cell following exposure to a compound, wherein said plurality of expression datasets comprises an expression dataset for each of a plurality of test compounds having a similar or identical biological activity, and an expression dataset for each of a plurality of control compounds that lack the biological activity of the test compounds; ii) deriving a discrimination metric that distinguishes the plurality of test compounds from the control compounds based on gene expression to provide a distinctive gene set; iii) selecting a plurality of genes from said distinctive gene set to provide a Group Signature for said plurality of test compounds; and iv) repeating steps i)-iii) for each Group Signature; b) contacting a subject cell with said drug candidate; c) extracting mRNA from said subject cell; d) reverse-transcribing said mRNA to cDNA; e) contacting said Group Signature Array with said cDNA; and f) determining whether any Group Signature probe set exhibits increased binding of cDNA. The invention also includes applying this method to a library of compounds and selecting a drug candidate, wherein the Group Signature probe set exhibits increased binding to the cDNA resulting from contacting the subject cell with said drug candidate.

Another aspect of the invention is a polynucleotide probe set for detecting fibrate-like activity, the set comprising: a plurality of polynucleotides capable of hybridizing specifically to genes selected from the group consisting of Rat for cytochrome P452, Rat cytochrome P450, Rat cytochrome P450-LA-omega (lauric acid omega-hydroxylase), Rat Sulfotransferase K2, Rat cytochrome P450-LA-omega (lauric acid omega-hydroxylase), Rat Cyp4a locus, encoding cytochrome P450 (IVA3), Rat cytochrome P450, Rat mitochondrial 3-2-trans-enoyl-CoA isomerase, Rat carnitine octanoyltransferase, Rat Wistar peroxisomal enoyl hydratase-like protein (PXEL), Rat mitochondrial long-chain 3-ketoacyl-CoA thiolase β-subunit of mitochondrial trifunctional protein, Rat liver fatty acid binding protein (FABP), Rat pyruvate dehydrogenase kinase isoenzyme 4 (PDK4), Rat mitochondrial isoform of cytochrome b5, Hypothetical protein Rv3224, Rat peroxisomal enoyl-CoA: hydrotase-3-hydroxyacyl-CoA bifunctional enzyme, Rat peroxisomal membrane protein Pmp26p (Peroxin-11), Rat acyl-CoA hydrolase, Rat acyl-CoA oxidase, Rat acyl-CoA hydrolase, Rat 2,4-dienoyl-CoA reductase precursor, Rat mitochondrial 3-hydroxy-3-methylglutaryl-CoA synthase, Rat peroxisomal enoyl-CoA: hydrotase-3-hydroxyacyl-CoA bifunctional enzyme, and Mouse peroxisomal long chain acyl-CoA thioesterase Ib (Pte1b).

Another aspect of the invention is a polynucleotide probe set for detecting gemfibrozil-like activity, the set comprising: a plurality of polynucleotides capable of hybridizing specifically to genes selected from the group consisting of Rat fatty acid synthase, Rat cholesterol 7α-hydroxylase, Mouse acetyl-CoA synthetase, Mouse Vanin-1, Rat kidney-specific protein (KS), Rat 2,3-oxidosqualene:lanosterol cyclase, Rat aldehyde dehydrogenase, and Rat thymosin β-10.

Another aspect of the invention is a method for screening drug candidates for fibrate activity, the method comprising: a) contacting a subject cell with a drug candidate; b) extracting mRNA from said subject cell; c) reverse-transcribing said mRNA into cDNA; d) hybridizing said cDNA to a fibrate signature probe set, said probe set comprising a plurality of polynucleotides capable of hybridizing specifically to a fibrate signature gene, wherein said fibrate signature genes are selected from the group consisting of Rat for cytochrome P452, Rat cytochrome P450, Rat cytochrome P450-LA-omega (lauric acid omega-hydroxylase), Rat Sulfotransferase K2, Rat cytochrome P450-LA-omega (lauric acid omega-hydroxylase), Rat Cyp4a locus, encoding cytochrome P450 (IVA3), Rat cytochrome P450, Rat mitochondrial 3-2-trans-enoyl-CoA isomerase, Rat carnitine octanoyltransferase, Rat Wistar peroxisomal enoyl hydratase-like protein (PXEL), Rat mitochondrial long-chain 3-ketoacyl-CoA thiolase β-subunit of mitochondrial trifunctional protein, Rat liver fatty acid binding protein (FABP), Rat pyruvate dehydrogenase kinase isoenzyme 4 (PDK4), Rat mitochondrial isoform of cytochrome b5, Hypothetical protein Rv3224, Rat peroxisomal enoyl-CoA: hydrotase-3-hydroxyacyl-CoA bifunctional enzyme, Rat peroxisomal membrane protein Pmp26p (Peroxin-11), Rat acyl-CoA hydrolase, Rat acyl-CoA oxidase, Rat acyl-CoA hydrolase, Rat 2,4-dienoyl-CoA reductase precursor, Rat mitochondrial 3-hydroxy-3-methylglutaryl-CoA synthase, Rat peroxisomal enoyl-CoA: hydrotase-3-hydroxyacyl-CoA bifunctional enzyme, and Mouse peroxisomal long chain acyl-CoA thioesterase Ib (Pte1b); and e) determining if said subject cell exhibits increased expression of a fibrate signature gene.

Another aspect of the invention is a database product, comprising: a computer-readable medium, said medium storing thereon a Group Signature database, said database comprising a plurality of Group Signature records, wherein each Group Signature record comprises indicia of at least one compound, wherein all compounds within a Group exhibit a similar or identical primary bioactivity; and indicia of a set of genes, wherein expression of said gene is modulated in response to exposure to a compound having a primary bioactivity similar or identical to the primary bioactivity of a compound indicated in the Group record, and wherein said set of genes distinguishes said Group from all other Groups within said Group Signature database.

### BRIEF DESCRIPTION OF THE FIGURES

FIG. 1 is a projection of a Principal Component Analysis output, showing the grouping of fibrate compounds along PCA1, split into male and female subjects along PCA2, and distinguished from octylphenol along PCA3.

FIG. 1A and FIG. 1B are rotated views of the same data.

FIG. 2 is a graph illustrating the specificity of a fenofibrate Drug Signature. The Drug Signature was based on four fenofibrate experiments compared to four control/vehicle experiments, and was then used to sort 677 other experiments. The sort was according to the similarity score S=ΠₓRelRkₓ. The sorted list was then graphed, assigning a value of 1.0 to each fenofibrate experiment, a value of 0.5 to each fibrate other than fenofibrate, and a value of 0 to each non-fibrate control. The graph demonstrates that this minimal fenofibrate Drug Signature correctly sorts most fenofibrate experiments to the top of the list, most fibrate experiments near the top of the list (although lower than fenofibrate experiments), and all control experiments below the fenofibrate experiments (and below most of the fibrate experiments).

FIG. 3 graphically presents bioassay results for seven nuclear receptor agonists (z axis from front to back: estradiol, bisphenol A, clofibrate, bis(2-ethyl-hexyl)phthalate (DEHP), fenofibrate, gemfibrozil, and octylphenol). Bioassays were selected from a panel of 123 assays performed if any of the selected compounds demonstrated activity: the 26 selected bioassays were (x axis) acetylcholinesterase (a); adenosine A2A (b); adenosine A3 (c); adrenergic α1D (d); adrenergic α2B (e); adrenergic α2C (f); adrenergic β3 (g); norepinephrine transporter (h); calcium channel type L (i); cyclooxygenase COX-2 (j); dopamine transporter (k); estrogen receptor (l); glucocorticoid receptor (m); lipoxygenase 15-LO (n); muscarinic receptor M1 (o); muscarinic receptor M2 (p); muscarinic receptor M3 (q); S/T kinase p38α (r); Y kinase EGF receptor (s); serotonin 5-HT2A (t); serotonin 5-HT2C (u); serotonin transporter (v); sodium channel-site 2 (w); tachykinin NK2 (x); testosterone receptor (y); thromboxane synthetase (z). The activity is shown as 1/IC₅₀ (y axis), with all values <50% inhibition binned to 0.

### DETAILED DESCRIPTION

Definitions:

The term “test compound” refers in general to a compound to which a test cell is exposed, about which one desires to collect data. Typical test compounds will be small organic molecules, typically drugs and/or prospective pharmaceutical lead compounds, but can include proteins, peptides, polynucleotides, heterologous genes (in expression systems), plasmids, polynucleotide analogs, peptide analogs, lipids, carbohydrates, viruses, phage, parasites, and the like.

The term “control compound” refers to a compound that is not known to share any biological activity with a test compound, which is used in the practice of the invention to contrast “active” (test) and “inactive” (control) compounds during the derivation of Group Signatures and Drug Signatures. Typical control compounds include, without limitation, drugs used to treat disorders distinct from the test compound indications, vehicles, known toxins, known inert compounds, and the like.

The term “biological activity” as used herein refers to the ability of a test compound to affect a biological system, for example to modulate the effect of an enzyme, block a receptor, stimulate a receptor, alter the expression of one or more genes, and the like. Test compounds have similar or identical biological activity when they have similar or identical effects on an organism in vivo or on cells or proteins in vitro. For example, fenofibrate, clofibrate, and gemfibrozil have similar biological activities because all three are prescribed for hyperlipoproteinemia. Similarly, aspirin, ibuprofen, and naproxen all have similar activities as all three are known to be non-steroidal anti-inflammatory compounds. The terms “primary bioactivity” and “primary biological activity” refer to the most pronounced or intended effect of the compound. For example, the primary bioactivity of an ACE inhibitor is the inhibition of angiotensin-converting enzyme (and the concomitant reduction of blood pressure), regardless of secondary bioactivities or side effects.

The term “subject cell” refers to a biological cell or a model of a biological system capable of reacting to the presence of a test compound, typically a live animal, eukaryotic cell or tissue sample, or a prokaryotic organism.

The term “expression response” refers to the change in expression level (if any) of a gene in response to administration of a test compound or control compound (or other test or control condition). The expression level can be measured directly, for example by quantifying the amount of protein encoded by the gene that is produced using proteomic techniques. A variety of methods for detecting protein levels may be used, including, but limited to, Western blots and ELISA. The expression level can also measured as the change in mRNA transcription, or by any other quantitative means of measuring gene activation. The expression response can be weighted or scaled as necessary to normalize data, and can be reported as the absolute increase or decrease in expression (or transcription), the relative change (for example, the percentage change), the degree of change above a threshold level, and the like.

The term “expression dataset” as used herein refers to data indicating the identity of genes affected by administration of the test or control compound, and the change in expression that resulted. The expression dataset typically contains a subset of genes, preferably the subset of genes that displayed the greatest changes in expression response.

The term “discrimination metric” refers to a method or algorithm for distinguishing the expression data in response to test compounds from the expression data in response to control compounds. The method can be selecting genes on the basis of the eigenvalues for the genes from the PCA output (selecting the principal component axis that separates the test compounds from the control compounds), or can include mathematical analysis to determine which gene or combination of genes best discriminates between the test and control compounds, for example using Golub's distinction metric, Student's t-test or the like.

The terms “PCA” and “principal component analysis” refer to mathematical methods for transforming a number of correlated variables into a number of uncorrelated (independent) variables called principal components. The first principal component accounts for as much of the variability in the data as possible, and each succeeding component accounts for as much of the remaining variability as possible. “PCA” as used herein further includes variations of principal component analysis such as kernel PCA and the like.

The term “Group Signature” as used herein refers to a data structure comprising a group identifier and one or more gene identifiers. The group identifier indicates a family of compounds having similar activity (for example, “fibrates”), or can directly indicate the activity (for example, PPARα inhibition). It is often simply the “name” of the group. The group identifier can further indicate the identity of compounds known to belong to the group. Gene identifiers indicate which gene expression rates are modulated (upregulated or downregulated) by exposure to a compound belonging to the group, and which are so characteristic of the group, or so distinctive, that modulation of the expression of these genes according to the signature is sufficient to distinguish the compound administered as belonging to the Group (rather than to another Group, or wholly lacking known activity). The gene identifiers can identify genes by sequence, name, reference to an accession number, reference to a clone or position within a DNA array, and the like. Gene identifiers can further comprise the direction and degree of expression modulation, in absolute or relative terms. For example, a gene identifier can include the requirement that expression decrease by at least 10%, or that expression increase by between 100% and 500%. The gene identifier can further include time restrictions: for example, a Group Signature can require that gene “X” be upregulated by at least 250% within 8 hours of administration, or at not less than 4 hours but no more than 16 hours, or the like. Although the Group Signature may comprise any number of genes, it typically comprises up to 50 gene identifiers of varying degrees of specificity, from which subsets of varying specificity can be derived. Preferably, the Group Signature consists of no more than 50 genes. More preferably, the Group Signature consists of no more than 25 genes. In addition, the Group Signature will comprise preferably at least three genes, more preferably at least 5 genes, even more preferably at least 10 genes and most preferably at least 15 genes. In some cases the Group Signature may consist of three or fewer. For example, the most specific signature for one group may comprise 20 gene identifiers: this signature contains a plurality of sub-signatures having similar (or somewhat less) specificity derived by omitting one or more of the gene identifiers. The Group Signature can further comprise bioassay data, for example indicating the bioactivity observed for compounds in the group against a panel of standard assays. Bioassay data can be used to identify the potential members of a Group prior to genomic experiments, particularly where a number of drug candidates are to be screened. Bioactivity data is particularly useful for distinguishing between compounds having unrelated structures, but which induce similar genomic expression patterns. The data structure can be stored physically or electronically, for example within a database on a computer-readable medium. Alternatively, the data structure can be embodied in an array in full or in part, such as a polynucleotide probe array having a separate region of probes specific for each Group Signature.

The term “Group Signature database” refers to a collection of data comprising a plurality of Group Signatures. A number of formats exists for storing data sets and simultaneously associating related attributes, including without limitation, tabular, relational, and dimensional. The tabular format is most familiar, for example spreadsheets such as Microsoft Excel® and Corel Quattro Pro® spreadsheets. In this format, association of data points with related attributes occurs by entering a data point and attributes related thereto in a unique row. Relational databases typically support a set of operations defined by relational algebra. Such databases typically include tables composed of columns and rows for the data included in the database. Each table in the database has a primary key, which can be any column or set of columns, the values for which uniquely identify the rows in the table. The tables in a relational database can also include a foreign key that is a column or set of columns, the values of which match the primary key values of another table. Typically, relational databases support a set of operations (for example, select, join, combine) that form the basis of the relational algebra governing relations within the database. Suitable relational databases include, without limitation, Oracle® (Oracle Inc., Redwood Shores, Calif.) and Sybase® (Sybase Systems, Emeryville, Calif.) databases.

The term “Drug Signature” as used herein refers to a data structure similar to the Group Signature, but specific to a single compound (or a plurality of essentially identical compounds, such as salts or esters of the same compound). The gene identifiers of a Drug Signature are selected to distinguish the selected compound from other compounds with which it shares activity(ies): Drug Signatures distinguish between members of a Group Signature, and also distinguish between the drug compound and unrelated compounds.

The term “gene expression profile” refers to a representation of the expression level of a plurality of genes in response to a selected expression condition (for example, incubation in the presence of a standard compound or test compound). Gene expression profiles can be expressed in terms of an absolute quantity of mRNA transcribed for each gene, as a ratio of mRNA transcribed in a test cell as compared with a control cell, and the like. As used herein, a “standard” gene expression profile refers to a profile already present in the primary database (for example, a profile obtained by incubation of a test cell with a standard compound, such as a drug of known activity), while a “test” gene expression profile refers to a profile generated under the conditions being investigated. The term “modulated” refers to an alteration in the expression level (induction or repression) to a measurable or detectable degree, as compared to a pre-established standard (for example, the expression level of a selected tissue or cell type at a selected phase under selected conditions).

The term “correlation information” as used herein refers to information related to a set of results. For example, correlation information for a profile result can comprise a list of similar profiles (profiles in which a plurality of the same genes are modulated to a similar degree, or in which related genes are modulated to a similar degree), a list of compounds that produce similar profiles, a list of the genes modulated in said profile, a list of the diseases and/or disorders in which a plurality of the same genes are modulated in a similar fashion, and the like. Correlation information for a compound-based inquiry can comprise a list of compounds having similar physical and chemical properties, compounds having similar shapes, compounds having similar biological activities, compounds that produce similar expression array profiles, and the like. Correlation information for a gene- or protein-based inquiry can comprise a list of genes or proteins having sequence similarity (at either nucleotide or amino acid level), genes or proteins having similar known functions or activities, genes or proteins subject to modulation or control by the same compounds, genes or proteins that belong to the same metabolic or signal pathway, genes or proteins belonging to similar metabolic or signal pathways, and the like. In general, correlation information is presented to assist a user in drawing parallels between diverse sets of data, enabling the user to create new hypotheses regarding gene and/or protein function, compound utility, and the like. Product correlation information assists the user with locating products that enable the user to test such hypotheses, and facilitates their purchase by the user.

“Similar”, as used herein, refers to a degree of difference between two quantities that is within a preselected threshold. For example, two genes can be considered “similar” if they exhibit sequence identity of more than a given threshold, such as for example 20%. A number of methods and systems for evaluating the degree of similarity of polynucleotide sequences are publicly available, for example BLAST, FASTA, and the like. See also Maslyn et al. and Fujimiya et al., supra, incorporated herein by reference. The similarity of two profiles can be defined in a number of different ways, for example in terms of the number of identical genes affected, the degree to which each gene is affected, and the like. Several different measures of similarity, or methods of scoring similarity, can be made available to the user: for example, one measure of similarity considers each gene that is induced (or repressed) past a threshold level, and increases the score for each gene in which both profiles indicate induction (or repression) of that gene. We utilize a similarity score that takes into account, for each gene, the level of regulation achieved by that gene in the experimental profile relative to all other experiments in the dataset. For a given gene, one can rank its level of regulation in the experimental profile relative to all other profiles (Rkₓ). The relative rank (RelRkₓ=Rkₓ/n, where n=number of profiles) is that rank divided by total number of profiles. A similarity score may then be defined as the product of these relative ranks for all genes in the profile or S=Πₓ RelRkₓ. A small value of S reflects an experimental profile that matches a reference profile on multiple genes and where the amplitude of regulation for each gene is large. Similarity between a test profile and a signature can be determined using a variety of metrics, a preferred one being defined as S=Πₓ RelRkₓ. The similarity score may also be referred to as a “specificity score” as it measures how rare the match of the experimental to the reference profile is relative to the rest of the dataset. Other statistical methods are also applicable.

The term “hyperlink” as used herein refers to feature of a displayed image or text that provides information additional and/or related to the information already currently displayed when activated, for example by clicking on the hyperlink. An HTML HREF is an example of a hyperlink within the scope of this invention. For example, when a user queries the database of the invention and obtains an output such as a list of the genes most induced or repressed by a selected compound, one or more of the genes listed in the output can be hyperlinked to related information. The related information can be, for example, additional information regarding the gene, a list of compounds that affect gene induction in a similar way, a list of genes having a known related function, a list of bioassays for determining activity of the gene product, product information regarding such related information, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing non-nucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA.

As used herein, the term “probe” or “oligonucleotide probe” refers to a structure comprised of a polynucleotide, as defined above, that contains a nucleic acid sequence capable of hybridizing to a nucleic acid sequence present in the target nucleic acid analyte. The polynucleotide regions of probes may be composed of DNA, and/or RNA, and/or synthetic nucleotide analogs. Probes of dozens to several hundred bases long can be artificially synthesized using oligonucleotide synthesizing machines, or they may be derived from various types of DNA cloning. A probe can be single-stranded or double-stranded. Probes are useful in the detection, identification and isolation of particular gene sequences or fragments. It is contemplated that any probe used in the present invention can be labeled with a reporter molecule so that it is detectable using a detection system, such as, for example, ELISA, EMIT, enzyme-based histochemical assays, fluorescence, radioactivity, luminescence, spin labeling, and the like. The critical aspects are that the probe must contain a nucleic acid strand that is at least partially complementary to the target sequence to be detected, and the probe must be labeled so that its presence can be visualized.

The terms “hybridize” and “hybridization” refer to the formation of complexes between nucleotide sequences which are sufficiently complementary to form complexes via Watson-Crick base pairing. It will be appreciated that the hybridizing sequences need not have perfect complementarity to provide stable hybrids. Further, the ability of two oligonucleotides to hybridize will be dependent on the experimental conditions. For example, the temperature and/or salt concentration will affect the percentage of complementary base pair matches required for hybrid duplexes to remain intact. Conditions that favor hybridization are referred to as less “stringent” than conditions that require a greater degree of sequence complementarity to maintain a stable duplex. In many situations, stable hybrids will form where fewer than about 10% of the bases are mismatches, ignoring loops of four or more nucleotides. Accordingly, as used herein the term “capable of hybridizing” refers to an oligonucleotide that can form a stable duplex with its “complement” under appropriate assay conditions, generally where there is about 90% or greater homology.

The terms “array”, “polynucleotide array”, “microarray”, and “probe array” all refer to a surface on which is attached or deposited a molecule capable of specifically binding a polynucleotide of a given sequence. Typically the molecule will be a polynucleotide having a sequence complementary to the polynucleotide to be detected, and capable of hybridizing to it.

General Method:

The method of the invention employs chemogenomic expression data and bioassay data in order to characterize and predict the biological activity of compounds. The method of the invention provides a way to cluster expression data meaningfully, and to extract relevant information from the sea of data that typically results from a genomic expression experiment.

The invention is based on the use of chemogenomic expression data, collected in response to an experimental condition, preferably contact with a compound or bioactive substance. Suitable compounds include known pharmaceutical agents, known and suspected toxins and pollutants, proteins, dyes and flavors, nutrients, herbal preparations, environmental samples, and the like. Other useful experimental conditions to examine include infectious agents such as viruses, bacteria, fungi, parasites, and the like, environmental stresses such as starvation, hypoxia, temperature, and the like. It is presently preferred to analyze a variety of compounds and/or experimental conditions simultaneously, particularly where many of the compounds and/or conditions are related by activity or therapeutic effect. The experimental conditions are applied to a cell having a genome, preferably a mammalian cell. Eukaryotic cells can be tested either in vivo or in vitro. Suitable eukaryotic cells include, without limitation, human, rat, mouse, cow, sheep, dog, cat, chicken, pig, goat, and the like. It is presently preferred to examine mammalian cells derived from a plurality of different tissue types, for example, liver, kidney, bone marrow, spleen, and the like. The subject cells are preferably exposed to a plurality of experimental conditions, for example, to a plurality of different concentrations of a compound, and examined at a plurality of time points.

The chemogenomic response can be obtained by any available means, for example by employing a panel of reporter cells, each group of cells having a reporter gene operatively connected to a different selected regulatory region. Alternatively, one can employ primary tissue isolates, cells or cell lines lacking reporter genes, and can determine the expression of a plurality of genes directly.

Direct detection methods include direct hybridization of mRNA with oligonucleotides or longer DNA fragments such as cDNA or even fragments of cloned genomic DNA (whether in solution or bound to a solid phase), reverse transcription followed by detection of the resulting cDNA, Northern blot analysis, and the like.

Primers and probes for use in the determination of expression levels herein are derived from gene sequences and are readily synthesized by standard techniques, e.g., solid phase synthesis via phosphoramidite chemistry, as disclosed in U.S. Pat. Nos. 4,458,066 and 4,415,732, incorporated herein by reference; Beaucage et al. (1992) Tetrahedron 48:2223-2311; and Applied Biosystems User Bulletin No. 13 (Apr. 1, 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly(A) or poly(C), or other non-complementary nucleotide extensions may be incorporated into probes using these same methods. Hexaethylene oxide extensions may be coupled to probes by methods known in the art. Cload et al. (1991) J. Am. Chem. Soc. 113:6324-6326; U.S. Pat. No. 4,914,210 to Levenson et al.; Durand et al. (1990) Nucleic Acids Res. 18:6353-6359; and Horn et al. (1986) Tet. Lett. 27:4705-4708.

While the length of the primers and probes can vary, the probe sequences are selected such that they have a lower melt temperature than the primer sequences. Hence, the primer sequences are generally longer than the probe sequences. Typically, the primer sequences are in the range of between 10-75 nucleotides long, more typically in the range of 20-45. The typical probe is in the range of between 10-50 nucleotides long, such as 15-40, 18-30, and so on, and any length between the stated ranges.

If a solid support is used, the oligonucleotide probe may be attached to the solid support in a variety of manners. For example, the probe may be attached to the solid support by attachment of the 3′ or 5′ terminal nucleotide of the probe to the solid support. More preferably, the probe is attached to the solid support by a linker which serves to distance the probe from the solid support. The linker is usually at least 15-30 atoms in length, more preferably at least 15-50 atoms in length. The required length of the linker will depend on the particular solid support used. For example, a six atom linker is generally sufficient when highly cross-linked polystyrene is used as the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred.

The linkages between the solid support, the linker and the probe are preferably not cleaved during removal of base protecting groups under basic conditions at high temperature. Examples of preferred linkages include carbamate and amide linkages. Examples of preferred types of solid supports for immobilization of the oligonucleotide probe include controlled pore glass, glass plates, polystyrene, avidin-coated polystyrene beads, cellulose, nylon, acrylamide gel and activated dextran.

Moreover, the probes may be coupled to labels for detection. As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens) and the like. The term “fluorescer” refers to a substance or a portion thereof which is capable of exhibiting fluorescence in the detectable range. There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al. Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly (1987) Nucl. Acids Res. 15:3131-3139, Gibson et al. (1987) Nucl. Acids Res. 15:6455-6467 and U.S. Pat. No. 4,605,735 to Miyoshi et al. Methods are also available for synthesizing sulfhydryl-derivatized oligonucleotides which can be reacted with thiol-specific labels, see, e.g., U.S. Pat. No. 4,757,141 to Fung et al., Connolly et al. (1985) Nucl. Acids Res. 13:4485-4502 and Spoat et al. (1987) Nucl. Acids Res. 15:4837-4848. A comprehensive review of methodologies for labeling DNA fragments is provided in Matthews et al., Anal. Biochem. (1988) 169:1-25.

Probes may be fluorescently labeled by linking a fluorescent molecule to the non-ligating terminus of the probe. Guidance for selecting appropriate fluorescent labels can be found in Smith et al., Meth. Enzymol. (1987) 155:260-301; Karger et al., Nucl. Acids Res. (1991) 19:4955-4962; Haugland (1989) Handbook of Fluorescent Probes and Research Chemicals (Molecular Probes, Inc., Eugene, Oreg.). Preferred fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Additionally, probes can be labeled with an acridinium ester (AE) using the techniques described below. Current technologies allow the AE label to be placed at any location within the probe. See, e.g., Nelson et al. (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

It is presently preferred to measure the genomic response by means of a nucleotide array, such as, for example, GeneChip® probe arrays (Affymetrix Inc., Santa Clara, Calif.), CodeLink™ Bioarray (Motorola Life Sciences, Northbrook, Ill.), and the like. Polynucleotide probes for interrogating the tissue or cell sample are preferably of sufficient length to specifically hybridize only to appropriate, complementary genes or transcripts. Typically, the polynucleotide probes used for this method will be at least 10, 12, 14, 16, 18, 20 or 25 nucleotides in length. In some cases, longer probes of at least 30, 40, or 50 nucleotides will be desirable. The genes examined using the array can comprise all of the genes present in the organism, or a subset of sufficient size to distinguish the genomic expression modulation due to compounds to the degree of resolution and/or confidence desired. The method of the invention is also useful for determining the size of a sufficient subset of genes necessary for this purpose.

One can employ target amplification methods (for example, PCR amplification of cDNA using Taqman® polymerase, and other enzymatic methods) and/or signal amplification methods (for example, employing highly-labeled probes, chromogenic enzymes, and the like) to determine the expression of the plurality of genes. Transcription-mediated amplification (TMA) is described in detail in, e.g., U.S. Pat. No. 5,399,491, the disclosure of which is incorporated herein by reference in its entirety. In one example of a typical assay, an isolated nucleic acid sample is mixed with a buffer concentrate containing the buffer, salts, magnesium, nucleotide triphosphates, primers, dithiothreitol, and spermidine. The reaction is optionally incubated at about 100° C. for approximately two minutes to denature any secondary structure. After cooling to room temperature, reverse transcriptase, RNA polymerase, and RNase H are added and the mixture is incubated for two to four hours at 37° C. The reaction can then be assayed by denaturing the product, adding a probe solution, incubating 20 minutes at 60° C., adding a solution to selectively hydrolyze the unhybridized probe, incubating the reaction six minutes at 60° C., and measuring the remaining chemiluminescence in a luminometer.

TMA provides a method of identifying target nucleic acid sequences present in very small amounts in a biological sample. Such sequences may be difficult or impossible to detect using direct assay methods. In particular, TMA is an isothermal, autocatalytic nucleic acid target amplification system that can provide more than a billion RNA copies of a target sequence. The assay can be done qualitatively, to accurately detect the presence or absence of the target sequence in a biological sample. The assay can also provide a quantitative measure of the amount of target sequence over a concentration range of several orders of magnitude. TMA provides a method for autocatalytically synthesizing multiple copies of a target nucleic acid sequence without repetitive manipulation of reaction conditions such as temperature, ionic strength and pH.

Generally, TMA includes the following steps: (a) isolating nucleic acid, including RNA, from the biological sample of interest; and (b) combining into a reaction mixture (i) the isolated nucleic acid, (ii) first and second oligonucleotide primers, the first primer having a complexing sequence sufficiently complementary to the 3′ terminal portion of an RNA target sequence, if present (for example the (+) strand), to complex therewith, and the second primer having a complexing sequence sufficiently complementary to the 3′ terminal portion of the target sequence of its complement (for example, the (−) strand) to complex therewith, wherein the first oligonucleotide further comprises a sequence 5′ to the complexing sequence which includes a promoter, (iii) a reverse transcriptase or RNA and DNA dependent DNA polymerases, (iv) an enzyme activity which selectively degrades the RNA strand of an RNA-DNA complex (such as an RNase H) and (v) an RNA polymerase which recognizes the promoter.

The components of the reaction mixture may be combined stepwise or at once. The reaction mixture is incubated under conditions whereby an oligonucleotide/target sequence hybrid is formed, including DNA priming and nucleic acid synthesizing conditions (including ribonucleotide triphosphates and deoxyribonucleotide triphosphates) for a period of time sufficient to provide multiple copies of the target sequence. The reaction advantageously takes place under conditions suitable for maintaining the stability of reaction components such as the component enzymes and without requiring modification or manipulation of reaction conditions during the course of the amplification reaction. Accordingly, the reaction may take place under conditions that are substantially isothermal and include substantially constant ionic strength and pH. The reaction conveniently does not require a denaturation step to separate the RNA-DNA complex produced by the first DNA extension reaction.

Suitable DNA polymerases include reverse transcriptases, such as avian myeloblastosis virus (AMV) reverse transcriptase (available from, e.g., Seikagaku America, Inc.) and Moloney murine leukemia virus (MMLV) reverse transcriptase (available from, e.g., Bethesda Research Laboratories).

Promoters or promoter sequences suitable for incorporation in the primers are nucleic acid sequences (either naturally occurring, produced synthetically or a product of a restriction digest) that are specifically recognized by an RNA polymerase that recognizes and binds to that sequence and initiates the process of transcription whereby RNA transcripts are produced. The sequence may optionally include nucleotide bases extending beyond the actual recognition site for the RNA polymerase which may impart added stability or susceptibility to degradation processes or increased transcription efficiency. Examples of useful promoters include those which are recognized by certain bacteriophage polymerases such as those from bacteriophage T3, T7 or SP6, or a promoter from E. coli. These RNA polymerases are readily available from commercial sources, such as New England Biolabs and Epicentre.

Some of the reverse transcriptases suitable for use in the methods herein have an RNase H activity, such as AMV reverse transcriptase. It may, however, be preferable to add exogenous RNase H, such as E. coli RNase H, even when AMV reverse transcriptase is used. RNase H is readily available from, e.g., Bethesda Research Laboratories.

The RNA transcripts produced by these methods may serve as templates to produce additional copies of the target sequence through the above-described mechanisms. The system is autocatalytic and amplification occurs autocatalytically without the need for repeatedly modifying or changing reaction conditions such as temperature, pH, ionic strength or the like.

As mentioned above, the primers and probes described above may be used in polymerase chain reaction (PCR)-based techniques to determine the expression levels of the plurality of genes. PCR is a technique for amplifying a desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. The polynucleotide sample is extracted and denatured, prefer-ably by heat, and hybridized with first and second primers which are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, two additional long products are produced, and a number of short products equal to the number of long and short products remaining at the end of the previous cycle. Thus, the number of short products containing the target sequence grow exponentially with each cycle. Preferably, PCR is carried out with a commercially available thermal cycler, e.g., Perkin Elmer.

RNAs may be amplified by reverse transcribing the mRNA into cDNA, and then performing PCR (RT-PCR), as described above. Alternatively, a single enzyme may be used for both steps as described in U.S. Pat. No. 5,322,770. mRNA may also be reverse transcribed into cDNA, followed by asymmetric gap ligase chain reaction (RT-AGLCR) as described by Marshall et al. (1994) PCR Meth. App. 4:80-84.

An alternate method, the fluorogenic 5′ nuclease assay, known as the TaqMan™ assay (Perkin-Elmer), is a powerful and versatile PCR-based detection system for nucleic acid targets. Hence, primers and probes can be used in TaqMan™ analyses. Analysis is performed in conjunction with thermal cycling by monitoring the generation of fluorescence signals. The assay system dispenses with the need for gel electrophoretic analysis, and has the capability to generate quantitative data allowing the determination of target copy numbers.

The fluorogenic 5′ nuclease assay is conveniently performed using, for example, AmpliTaq Gold™ DNA polymerase, which has endogenous 5′ nuclease activity, to digest an internal oligonucleotide probe labeled with both a fluorescent reporter dye and a quencher (see, Holland et al., Proc. Natl. Acad. Sci. USA (1991) 88:7276-7280; and Lee et al., Nucl. Acids Res. (1993) 21:3761-3766). Assay results are detected by measuring changes in fluorescence that occur during the amplification cycle as the fluorescent probe is digested, uncoupling the dye and quencher labels and causing an increase in the fluorescent signal that is proportional to the amplification of target DNA. For a detailed description of the TaqMan™ assay, reagents and conditions for use therein, see, e.g., Holland et al., Proc. Natl. Acad. Sci, U.S.A. (1991) 88:7276-7280; U.S. Pat. Nos. 5,538,848, 5,723,591, and 5,876,930, all incorporated herein by reference in their entireties.

The amplification products can be detected in solution or using solid supports. In this method, the TaqMan™ probe is designed to hybridize to a target sequence within the desired PCR product. The 5′ end of the TaqMan™ probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. Excision of the 5′ fluorophore results in an increase in fluorescence which can be detected. In particular, the oligonucleotide probe is constructed such that the probe exists in at least one single-stranded conformation when unhybridized where the quencher molecule is near enough to the reporter molecule to quench the fluorescence of the reporter molecule. The oligonucleotide probe also exists in at least one conformation when hybridized to a target polynucleotide such that the quencher molecule is not positioned close enough to the reporter molecule to quench the fluorescence of the reporter molecule. By adopting these hybridized and unhybridized conformations, the reporter molecule and quencher molecule on the probe exhibit different fluorescence signal intensities when the probe is hybridized and unhybridized. As a result, it is possible to determine whether the probe is hybridized or unhybridized based on a change in the fluorescence intensity of the reporter molecule, the quencher molecule, or a combination thereof. In addition, because the probe can be designed such that the quencher molecule quenches the reporter molecule when the probe is not hybridized, the probe can be designed such that the reporter molecule exhibits limited fluorescence unless the probe is either hybridized or digested.

The Ligase Chain Reaction (LCR) is an alternate method for nucleic acid amplification and thus detection of expression levels. In LCR, probe pairs are used which include two primary (first and second) and two secondary (third and fourth) probes, all of which are used in molar excess to target. The first probe hybridizes to a first segment of the target strand, and the second probe hybridizes to a second segment of the target strand, the first and second segments being contiguous so that the primary probes abut one another in 5′ phosphate-3′ hydroxyl relationship. Thus, a ligase can covalently fuse or ligate the two probes into a fused product. In addition, a third (secondary) probe can hybridize to a portion of the first probe and a fourth (secondary) probe can hybridize to a portion of the second probe in a similar abutting fashion. If the target is initially double-stranded, the secondary probes will also hybridize to the target complement in the first instance. Once the ligated strand of primary probes is separated from the target strand, it will hybridize with the third and fourth probes which can be ligated to form a complementary, secondary ligated product. By repeated cycles of hybridization and ligation, amplification of the target sequence is achieved. This technique is described in, e.g., European Publication No. 320,308, published Jun. 16, 1989 and European Publication No. 439,182, published Jul. 31, 1991.

One preferable method of detecting the level of expression of a gene is the use of target sequence-specific oligonucleotide probes. The probes may be used in hybridization protection assays (HPA). In this embodiment, the probes are conveniently labeled with acridinium ester (AE), a highly chemiluminescent molecule. One AE molecule is directly attached to the probe using a non-nucleotide-based linker arm chemistry that allows placement of the label at any location within the probe. Chemiluminescence is triggered by reaction with alkaline hydrogen peroxide which yields an excited N-methyl acridone that subsequently collapses to ground state with the emission of a photon. Additionally, AE causes ester hydrolysis which yields the nonchemiluminescent-methyl acridinium carboxylic acid.

When the AE molecule is covalently attached to a nucleic acid probe, hydrolysis is rapid under mildly alkaline conditions. When the AE-labeled probe is exactly complementary to the target nucleic acid, the rate of AE hydrolysis is greatly reduced. Thus, hybridized and unhybridized AE-labeled probe can be detected directly in solution, without the need for physical separation.

HPA generally consists of the following steps: (a) the AE-labeled probe is hybridized with the target nucleic acid in solution for about 15 to about 30 minutes. A mild alkaline solution is then added and AE coupled to the unhybridized probe is hydrolyzed. This reaction takes approximately 5 to 10 minutes. The remaining hybrid-associated AE is detected as a measure of the amount of target present. This step takes approximately 2 to 5 seconds. Preferably, the differential hydrolysis step is conducted at the same temperature as the hybridization step, typically at 50 to 70° C. Alternatively, a second differential hydrolysis step may be conducted at room temperature. This allows elevated pHs to be used, for example in the range of 10-11, which yields larger differences in the rate of hydrolysis between hybridized and unhybridized AE-labeled probe. HPA is described in detail in, e.g., U.S. Pat. Nos. 6,004,745; 5,948,899; and 5,283,174, the disclosures of which are incorporated by reference herein in their entireties.

Nucleic acid sequence-based amplification (NASBA) may also be used in the present invention for determining the expression of a plurality of genes. This method is a promoter-directed, enzymatic process that induces in vitro continuous, homogeneous and isothermal amplification of a specific nucleic acid to provide RNA copies of the nucleic acid. The reagents for conducting NASBA include a first DNA primer with a 5′ tail comprising a promoter, a second DNA primer, reverse transcriptase, RNase-H, T7 RNA polymerase, NTP's and dNTP's. Using NASBA, large amounts of single-stranded RNA are generated from either single-stranded RNA or DNA, or double-stranded DNA. When RNA is to be amplified, the ssRNA serves as a template for the synthesis of a first DNA strand by elongation of a first primer containing an RNA polymerase recognition site. This DNA strand in turn serves as the template for the synthesis of a second, complementary, DNA strand by elongation of a second primer, resulting in a double-stranded active RNA-polymerase promoter site, and the second DNA strand serves as a template for the synthesis of large amounts of the first template, the ssRNA, with the aid of a RNA polymerase. The NASBA technique is known in the art and described in, e.g., Guatelli et al. (1990) Proc. Natl. Acad. Sci. USA 87:1874-1878; Compton, J. Nature 350:91-92; European Patent 329,822, International Patent Application No. WO 91/02814, and U.S. Pat. Nos. 6,063,603, 5,554,517 and 5,409,818, all of which are incorporated herein in their entireties.

Other known amplification and detection methods that can be utilized include, but are not limited to, Q-beta amplification; strand displacement amplification (Walker et al. Clin. Chem. 42:9-13 and European Patent Application No. 684,315); and target mediated amplification (International Publication No. WO 93/22461).

Many of the described methods rely on complementarity between a probe or primer and a target nucleic acid. When ssDNA molecules form hybrids, the base sequence complementarity of the two strands does not have to be perfect. Poorly matched hybrids (i.e., hybrids in which only some of the nucleotides in each strand are aligned with their complementary bases so as to be able to form hydrogen bonds) can form at low temperatures, but as the temperature is raised (or the salt concentration lowered) the complementary base-paired regions within the poorer hybrids dissociate due to the fact that there is not enough total hydrogen bond formation within the entire duplex molecule to hold the two strands together under the new environmental conditions. The temperature and/or salt concentrations may be changed progressively so as to create conditions where an increasing percentage of complementary base pair matches is required in order for hybrid duplexes to remain intact. Eventually, a set of conditions will be reached at which only perfect hybrids can exist as duplexes. Above this stringency level, even perfectly matched duplexes will dissociate. The stringency conditions for each unique fragment of dsDNA in a mixture of DNA depends on its unique base pair composition. The degree to which hybridization conditions require perfect base pair complementarity for hybrid duplexes to persist is referred to as the “stringency of hybridization.” Low stringency conditions are those which permit the formation of duplex molecules having some degree of mismatched bases. High stringency conditions are those which permit only near-perfect base pair-matched duplex molecules to persist. Manipulation of stringency conditions is key to the optimization of sequence specific assays. It will be appreciated that the present methods do not require perfect base-pair-matched duplexes.

More particularly, in the amplification-based methods described above, once the primers or probes have been sufficiently extended and/or ligated, they are separated from the target sequence, for example, by heating the reaction mixture to a “melt temperature” which dissociates the complementary nucleic acid strands. Thus, a sequence complementary to the target sequence is formed. A new amplification cycle can then take place to further amplify the number of target sequences by separating any double-stranded sequences, allowing primers or probes to hybridize to their respective targets, extending and/or ligating the hybridized primers or probes and re-separating. The complementary sequences that are generated by amplification cycles can serve as templates for primer extension or fill the gap of two probes to further amplify the number of target sequences. Typically, a reaction mixture is cycled between 20 and 100 times, more typically between 25 and 50 times. In this manner, multiple copies of the target sequence and its complementary sequence are produced. Thus, primers initiate amplification of the target sequence when it is present under amplification conditions.

The “melting temperature” or “Tm” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25° C. below the Tₘ. The Tₘ may be estimated using the following relationship: Tₘ=69.3+0.41(GC)% (Marmur et al. (1962) J. Mol. Biol. 5:109-118).

In another aspect of the invention, two or more of the tests described above are performed. For example, if the first test used the transcription mediated amplification (TMA) to amplify the nucleic acids for detection, then an alternative nucleic acid testing (NAT) assay is performed, for example, by using PCR amplification, RT PCR, and the like, as described herein. As is readily apparent, design of the assays described herein are subject to a great deal of variation, and many formats are known in the art. The above descriptions are merely provided as guidance and one of skill in the art can readily modify the described protocols, using techniques well known in the art.

Detection, both amplified and nonamplified, may be performed using a variety of heterogeneous and homogeneous detection formats. Examples of heterogeneous detection formats are disclosed in Snitman et al., U.S. Pat. No. 5,273,882; Urdea et al., U.S. Pat. No. 5,124,246; Ullman et al. U.S. Pat. No. 5,185,243; and Kourilsky et al., U.S. Pat. No. 4,581,333, all of which are incorporated herein by reference in their entireties. Examples of homogeneous detection formats are described in Caskey et al., U.S. Pat. No. 5,582,989; and Gelfand et al., U.S. Pat. No. 5,210,015, which are incorporated herein by reference in their entireties. Also contemplated and within the scope of the present invention is the use of multiple probes in hybridization assays, to improve sensitivity and amplification of the target signal. See, for example, Caskey et al., U.S. Pat. No. 5,582,989; and Gelfand et al., U.S. Pat. No. 5,210,015; which are incorporated herein by reference in their entireties.

Protocols have been developed to evaluate rapidly multiple candidate compounds in a particular system and/or a candidate compound in a plurality of systems. Such protocols for evaluating candidate compounds have been referred to as high throughput screening (HTS). In one typical protocol, HTS involves the dispersal of a candidate compound into a well of a multiwell cluster plate, for example, a 96-well or higher format plate, e.g., a 384-, 864-, or 1536-well plate. The effect of the compound is evaluated on the system in which it is being tested. The “throughput” of this technique, i.e., the combination of the number of candidate compounds that can be screened and the number of systems against which candidate compounds can be screened, is limited by a number of factors, including, but not limited to: only one assay can be performed per well; if conventional dye molecules are used to monitor the effect of the candidate compound, multiple excitation sources are required if multiple dye molecules are used; and as the well size becomes small (e.g., the 1536-well plate can accept about 5 μl of total assay volume), consistent dispensing of individual components into a well is difficult and the amount of signal generated by each assay is significantly decreased, scaling with the volume of the assay.

A 1536-well plate is merely the physical segregation of sixteen assays within a single 96 well plate format. It would be advantageous to multiplex 16 assays into a single well of the 96 well plate. This would result in greater ease of dispensing reagents into the wells and in high signal output per well. In addition, performing multiple assays in a single well allows simultaneous determination of the potential of a candidate compound to affect a plurality of target systems. Using HTS strategies, a single candidate compound can be screened for activity as, e.g., a protease inhibitor, an inflammation inhibitor, an anti-asthmatic, and the like, in a single assay.

In still another embodiment of the invention, an HTS assay using emission labels as multiplexed detection reagents is provided. The HTS assay is performed in the presence of various concentrations of a candidate compound. Emission is monitored as an indication of the effect of the candidate compound on the assay system. For example, fluorescence reading using a labeled ligand or receptor to monitor binding thereof to a bead-bound receptor or ligand, respectively, may be used as a flexible format to measure emission associated with the beads. The measure of emission associated with the beads can be a function of the concentration of candidate compound and, thus, of the effect of the candidate compound on the system. In addition, a multicolor scintillant can be used to detect the binding of a radiolabeled ligand or receptor with a labeled receptor or ligand, respectively. A decrease in scintillation would be one result of inhibition by the candidate compound of the ligand-receptor pair binding. Thus a large number of genes can be evaluated using HTS techniques to prepare expression datasets.

The data obtained, whether resulting from the array experiments or otherwise, is generally expressed in terms of the amount or degree of gene expression, and whether it is significantly upregulated or down-regulated. The data may be subjected to one or more manipulations, for example to normalize data from an array (comparing data from points in different regions of the physical array, to adjust for systematic errors). Data is frequently presented in the form of a ratio, for example the experimental expression level compared to the control level, where the control level can be the untreated expression level for the same gene, a historical untreated level, a pooled expression level for a number of genes, and the like. Each data point is associated with a compound (or control), a gene or polynucleotide sequence corresponding to the mRNA detected, and an expression level, and can further comprise other experimental conditions such as, for example, time, temperature, subject animal species, subject animal gender, subject animal age, other treatment of the subject animal (such as fasting, stress, prior or concurrent administration of other compounds, time and manner of sacrifice, and the like), tissue or cell line from which the data is derived, type of array and serial number, date of experiment, researcher or client for whom the experiment was performed, and the like.

When examining datasets derived from several hundred or more genes, it is presently preferred to select the genes that exhibit the greatest variability in expression level during the experiment. We have found that for most compounds only a few genes respond to a high degree (for example, an increase in expression level by a factor of five or more), and approximately 100 to 500 exhibit a lesser but still substantial response. Most genes do not significantly respond, and can be excluded from the remainder of the analysis without loss of information. The observed variability in expression level can be adjusted for the available “dynamic range” of each gene: for example, if gene A exhibits a maximum change in expression level of only a factor of 2, and gene B exhibits a maximum change in expression level of a factor of 30, one expects that gene A at 2 is exhibiting a stronger relative response than gene B at 4. Accordingly, the genes can be selected based on the ratio of their observed variability (for example, standard deviation) to their possible variability (for example, the greatest degree of variation observed historically, for all experiments). It is presently preferred to order the genes by variability, and to select the 200 most variable genes for the remainder of the analysis.

It is typical for genomic expression experiments to present data in the form of a two-dimensional table or matrix, where each gene is allotted a row, and each column corresponds to an experiment or experimental condition. In contrast, the method of the invention allots a row to each compound as the row variable, and a column to each gene. The data records are then clustered by compound, thus grouping all compounds (and optionally by experimental conditions) on the basis of similar gene expression modulation. This permits one to directly identify which genes are most affected by the presence of the compounds used.

It is presently preferred to select a variety of related compounds (the “experimental group”), together with several compounds unrelated to the experimental group (“counter group”) for examination and analysis under a variety of experimental conditions, such as, for example, a plurality of time points post-administration. The compounds included in the experimental group are preferably related by virtue of having similar mechanisms of action (or are believed to act by the same pathway). For purposes of developing a group signature, it is presently preferred to select at least two compounds for the experimental group, at a plurality of different experimental conditions (for example, each compound examined at several time points). The maximum number of compounds that can be included in the experimental group is typically limited by the number of related compounds available, but in any case is preferably limited to no more than 200. The number of compounds included in the counter group is preferably at least two, more preferably at least 10, and preferably no more than 200, preferably less than 100, most preferably less than 50. Preferably, the counter group is selected so that it does not contain a group of related compounds larger than the number of related compounds in the experimental group.

The compounds are tested and the resulting data is treated as described above, and then preferably analyzed by principal component analysis (PCA) to determine the sets of treatments (experiments) that form resolvable clusters. Once it is established which treatments can form resolvable clusters one can determine the genes or groups of genes are most responsible for the observed effect of the compound. Several methods for achieving this goal are presented below. If the compounds selected for the experimental group are related by activity, their data points will form a distinct cluster in PCA analysis, separate from the data points belonging to the counter group (which may or may not form one or more clusters, depending on the compounds selected). The experimental group will typically dominate one PCA axis, with most or all of the counter group situated at lower values along the axis. The eigenvalues for the genes comprising the corresponding PCA axis can then be examined to determine which genes are modulated to the greatest degree by the experimental group: this group of genes provides a pool from which the Group Signature is determined. The Group Signature comprises a set of genes capable of distinguishing the group activity (the common biological activity exhibited by the compounds in the experimental group) from other activities. For example, the Group Signature obtained for fibrates in Example 1 below is capable of distinguishing between compounds having fibrate activity (such as clofibrate, fenofibrate, gemfibrozil, and the like) from compounds having other activities (such as estrogenic compounds, phenols, and the like). If the genes included in the PCA axis that corresponds to the experimental group activity are sorted and ranked by eigenvalue (in other words, in order of their contribution to that principal component), the genes that sort to the top of the list will comprise the Group Signature. The Group Signature need not include all of the genes ranked at the top, but should include at least the top three, and preferably further includes at least five of the top ten, more preferably at least 10 of the top 20 genes.

Alternatively, the Group Signature can be defined by performing a distinctiveness calculation to determine which genes distinguish the experimental group best from the counter group. For example, one can employ the distinction metric set forth by T. R. Golub et al., Science (1999) 286(5439):531-37, where distinction is calculated as

mean₁−mean₂/(stdev₁+stdev₂)

where mean₁ and stdev₁ refer to the mean expression level and standard deviation of expression levels for gene “1”. This calculation will generally produce a very similar (although not necessarily identical) set of genes for the Group Signature. It is presently preferred to use a modified form of the Golub metric, where distinction is calculated as

mean₁−mean₂/(stdev₁+stdev₂+0.01)

in order to avoid errors in cases where the standard deviation (stdev) terms in the denominator are zero or close to zero. This happens often by chance when a small number of experiments are used to define the groups. The problem is exacerbated when the data is filtered by a quality control metric and the ratios are reset to one (Log ratio=0). The small value of 0.01 added to the denominator can be modified for linear ratios (log₁₀ of the ratio is presently preferred).

If desired, the Group Signature can be further refined by comparing the expression patterns of two or more compounds at opposite ends of the PCA axis along which they spread, for example selecting a compound having a high degree of a known bioactivity and a second compound having a low degree of the same bioactivity. If the genes (already sorted for selection as part of the Group Signature) are then compared for variation between these two selected compounds, one can identify the genes that correlate most closely with the bioactivity of the compounds in the group.

It is sometimes helpful to examine the original data using PCA, to determine if any systematic errors are present. For example, if the data clusters according to experiment date, lab technician, or the like, further analysis of the data is typically warranted. It is useful to note that a systematic bias can occur that separates all treatments into subgroups (along a PCA axis for example), yet this does not preclude the detection and visualization of additional real effects. This capacity of PCA to group experiments in three dimensions, and thus to visualize multiple simultaneous effects including systematic biases, is a marked advantage compared to other methods such 2D hierarchical clustering, where a single dimension is used to cluster experiments and the other dimension is used to cluster the genes.

The similarity between an experimental treatment and a signature can be quantitated in a variety of ways. For example, in a signature consisting of upregulated genes A, B, and C, if the induction level for gene A in an experiment is reached (or surpassed) 1% of the time, the expression level for gene B is reached (or surpassed) 3% of the time, and the expression level for gene C is reached (or surpassed) 12% of the time, the specificity would be calculated 0.01×0.03×0.12=0.000036. If genes A, B, and C exhibited their expression levels more often, for example 4%, 6%, and 15% respectively, the resulting score would be lower (0.04×0.05×0.15=0.0003), because the gene expression levels would be less distinctive or characteristic. Generalizing this calculation for a signature of any length we obtain: S=Πₓ RelRkₓ where RelRkx is the relative rank as defined above. The score can be further refined by weighting each gene's contribution: the genes that are ranked lower in the signature are less important, and less distinctive than those ranked higher. Thus, for example, one can calculate a weighted specificity by dividing the probability score for each gene by its rank in the signature, or by a multiple or higher power of the rank. For example, given a signature consisting of upregulated genes X, Y, and Z, wherein the induction level for gene X is reached in 1% of the experiments, the induction level for gene Y is reached in 3% of the experiments, and the induction level for gene Z is reached in 12% of the experiments, a simple additive specificity would be 0.010+0.030+0.120=0.160. In a weighted specificity in which each term was divided by the gene rank, the specificity would be calculated (0.010/1)+(0.030/2)+(0.120/3)=0.065. A signature in which the first gene was less predictive (higher probability) would have a higher score (indicating less specificity): for example, if the probabilities for genes X, Y and Z were reversed, the same specificity would be calculated (0.120/1)+(0.030/2)+(0.010/3)=0.138. The specificity score can be weighted more heavily by increasing its dependence on gene rank, for example using the square or cube of the gene rank as the divisor. Thus, for example, the XYZ signature can be calculated as (0.010/1)+(0.030/4)+(0.120/9)=0.0308 using the square of the rank, or (0.010/1)+(0.030/8)+(0.120/27)=0.0182 using the cube of the rank. Again, comparing the results with the specificity scores obtained with the probabilities reversed (0.1286 and 0.1241, respectively), one can see that the difference in score increases with increased weighting: the difference in specificity score between XYZ and “reversed” XYZ is 0.0723 for weighting by rank, 0.0978 for weighting by the square of the rank, and 0.1059 for weighting by the cube of the rank. Alternatively, one can use other weighting factors, such as for example, the gene rank raised to a non-integral power (for example, 2.1, 2.5, 4.2, and the like), the logarithm of the rank, a set of arbitrarily-selected constants (for example, using as divisor 1, 2, 4, 8, and 10 for the first five genes, and 15 for each additional gene), and the like. One can use a power <1, such as square root (=½): this has the effect of decreasing the weight of the rank. This in effect allows weighting over a longer signature.

The Group Signature is useful for identifying the gene regulatory pathways most affected by the compounds in the experimental group, and by extension the genes most involved in the response to the compounds and/or the biological effect induced by the compounds, particularly when combined with bioassay information regarding the effect of the compounds on a variety of known enzymes and binding proteins.

The Group Signature is also useful for classifying or characterizing a new compound based on its genomic expression pattern, and predicting the potential therapeutic activity thereof. Comparing the expression pattern of several thousand genes in response to a compound with the expression patterns of several thousand genes to a large number of other compounds is a very calculation-intensive activity. However, one can compile a database of Group Signatures, having one or more signatures for each class of therapeutic compound (for example, a fibrate signature, an ACE inhibitor signature, a caspase inhibitor signature, and the like), where each signature need only include, for example, 10 to 20 gene expression patterns. The resulting Group Signature database is much smaller than a complete database of genomic expression patterns, and can be queried rapidly. Genes that have not been selected to comprise any Group Signature in the database need not be examined at all.

Further, Group Signatures can be directly “embodied” in a probe set (whether in a polynucleotide array or in solution phase) and other detection reagents. For example, a substrate can be provided with a plurality of group areas, each group area containing polynucleotide sequences capable of specifically binding sequences present in a specific Group Signature. Thus, a Group Signature Chip may have a first region containing probes specific for the fibrate Group Signature, a second region containing probes specific for the phenyl-acetic acid (for example, aspirin, naproxen, ibuprofen) Group Signature, and so forth. The probes for each Group Signature are preferably selected so that they do not overlap, or overlap to a minimal degree. Alternatively, if two or more Group Signatures include a common set of genes, the chip can be arranged to include probes for the common set as the intersection between two signatures, for example so that Signature 1 comprises region 1 plus common region X, and Signature 2 comprises region 2 plus common region X. The Group Signatures present on the chip can include both signatures from therapeutic drugs, and signatures of specific modes of toxicity. Thus, mRNA or cDNA can be obtained from a subject cell after exposure to a test compound, labeled, and applied directly to the Group Signature Chip: the activity(ies) and toxicity of the test compound (if any) is then identified directly by determining which Group Signatures exhibit binding.

The above-described assay reagents, including the primers, probes, solid support with bound probes, as well as other detection reagents, can be provided in kits, with suitable instructions and other necessary reagents, in order to conduct the assays as described above. The kit will normally contain in separate containers the combination of primers and probes (either already bound to a solid matrix or separate with reagents for binding them to the matrix), control formulations (positive and/or negative), labeled reagents when the assay format requires same and signal generating reagents (e.g., enzyme substrate) if the label does not generate a signal directly. Instructions (e.g., written, tape, VCR, CD-ROM, etc.) for carrying out the assay usually will be included in the kit. The kit can also contain, depending on the particular assay used, other packaged reagents and materials (i.e., wash buffers and the like). Standard assays, such as those described above, can be conducted using these kits.

Individual compounds can be examined to provide specific Drug Signatures capable of distinguishing between members of the same group (to the extent that the subject cells are capable of exhibiting a distinct response between the members). By selecting genes that distinguish a selected compound from other compounds in its group from the sorted list of genes from which the Group Signature is derived, one can obtain a Drug Signature that indicates how the subject cell responds differently to the selected compound. The Drug Signature is useful for identifying toxicities and side effects that are peculiar to the selected compound, as well as possible synergistic effects: i.e., the Drug Signature can be used to explain or determine why one compound has greater or lesser activity, and/or why one compound would be a better therapeutic choice for a particular patient (based on the patient's condition).

Fenofibrate, clofibrate, and gemfibrozil are fibric acid derivatives commonly-prescribed for treating hyperlipoproteinemia.

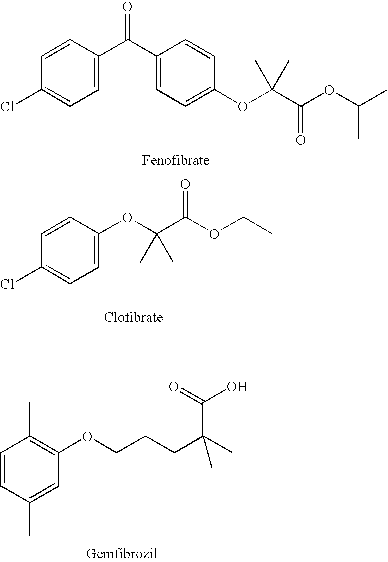

We have now determined a Group Signature for the fibrate group, which comprises an expression profile in which a combination of the genes set forth below are strongly upregulated:

Fibrate Group Signature

| Clone ID | Gene |
|---|---|
| 701507855 | Rat mRNA for cytochrome P452 |
| 700296865 | Rat cytochrome P450 mRNA, complete cds |
| 701466373 | Rat cytochrome P450-LA-omega (lauric acid omega- |
| | hydroxylase) mRNA, complete cds |
| 701197528 | Rat mRNA for Sulfotransferase K2 |
| 701444552 | Rat cytochrome P450-LA-omega (lauric acid omega- |
| | hydroxylase) mRNA, complete cds |
| 701196893 | Rat Cyp4a locus, encoding cytochrome P450 (IVA3) |
| | mRNA, complete cds |
| 700296634 | Rat cytochrome P450 mRNA, complete cds |
| 700481210 | Rat mRNA for mitochondrial 3-2-trans-enoyl-CoA |
| | isomerase |
| 701531239 | Rat carnitine octanoyltransferase mRNA, complete cds |
| 701880740 | Unnamed protein product |
| 700247611 | Rat Wistar peroxisomal enoyl hydratase-like protein |
| | (PXEL) mRNA, complete cds |
| 700397284 | Rat mRNA for mitochondrial long-chain 3-ketoacyl-CoA |
| | thiolase β-subunit of mitochondrial trifunctional protein, |
| | complete cds |
| 700505778 | Rat liver fatty acid binding protein (FABP) mRNA. |
| 700187344 | Rat pyruvate dehydrogenase kinase isoenzyme 4 (PDK4) |
| | mRNA, complete cds |
| 700935253 | Rat mRNA for mitochondrial isoform of cytochrome b5 |
| 701826047 | Hypothetical protein Rv3224 |
| 701512411 | Incyte EST |
| 700935113 | Rat peroxisomal enoyl-CoA: hydrotase-3-hydroxyacyl-CoA |
| | bifunctional enzyme mRNA, complete cds |
| 701512110 | Rat peroxisomal membrane protein Pmp26p (Peroxin-11) |
| 700146486 | Rat mRNA for acyl-CoA hydrolase, complete cds |
| 701646795 | Rat acyl-CoA oxidase mRNA, complete cds |
| 701466951 | Rat mRNA for acyl-CoA hydrolase, complete cds |
| 700628567 | Rat mRNA for 2,4-dienoyl-CoA reductase precursor, |
| | complete cds |
| 700199767 | Rat mitochondrial 3-hydroxy-3-methylglutaryl-CoA |
| | synthase mRNA, complete cds |
| 701469162 | Rat peroxisomal enoyl-CoA: hydrotase-3-hydroxyacyl-CoA |
| | bifunctional enzyme mRNA, complete cds |
| 701606788 | Mouse peroxisomal long chain acyl-CoA thioesterase Ib |
| | (Ptelb) gene, exon 3 and complete cds |

The fibrate Group Signature includes at least three of the genes listed, preferably at least three of the first five genes listed, more preferably at least five of the first ten genes listed, more preferably at least fifteen genes including at least seven of the first ten genes listed above, or their equivalents. The Group Signature preferably contains no more than 25 genes, more preferably from 20 to 25 genes. If desired, the Group Signature can be further refined by including time and dosage variation: for example, fibrate compounds at a given dosage may maximally stimulate expression of one gene at 12 hours, and of a different gene at 48 hours. The resulting refinements can be used to generate a more precise Group Signature.

The fibrate Group Signature is useful for identifying other compounds that have a biological activity similar or identical to the fibrates, i.e., that exhibit PPARα agonist activity. For example, a series of experimental compounds can be administered to rat liver tissue isolates at a variety of concentrations. At a variety of time points after administration, the liver cells are examined to determine which genes have been upregulated: for example, the total mRNA can be reverse-transcribed to provide cDNA, and the cDNA can then be subjected to hybridization with a set of polynucleotide probes, for example bound to a solid surface. The set of probes is selected to include polynucleotide sequences corresponding to the fibrate Group Signature: thus, any experimental compound that generates a strong signal (i.e., a signal that strongly matches the selected fibrate Group Signature) is identified as having PPARα agonist activity.

The fibrate Group Signature can further be used to design probe sets and reagents for the detection of fibrate drugs, and for screening compounds for potential PPARα activity. The fibrate Group Signature probes can be provided as part of a collection of Group Signature probes designed to detect a variety of similar or different activities. For example, one can provide a kit comprising 20 polynucleotide probes selected from the fibrate Group Signature alone, or alternatively one can provide a kit comprising that probe set in addition to one or more additional probe sets selected from other Group Signatures. The probe sets can further comprise additional probes, provided as controls and/or to detect other conditions, for example to monitor toxicity.

A distinct Drug Signature was derived for gemfibrozil, which is capable of distinguishing gemfibrozil from the other fibrate compounds. This signature was derived from the top ten distinctive genes that were upregulated in response to gemfibrozil:

| Clone ID | Gene |
|---|---|
| 700532842 | Unknown |
| 700290539 | Rat fatty acid synthase mRNA, complete cds |
| 701581809 | Incyte EST |
| 701436793 | Rat cholesterol 7α-hydroxylase gene, exon 6 |
| 700183232 | Mouse acetyl-CoA synthetase mRNA, complete cds |
| 700933512 | Mouse mRNA for Vanin-1 |
| 700304757 | Rat kidney-specific protein (KS) mRNA, complete cds |
| 701228305 | Rat mRNA for 2,3-oxidosqualene: lanosterol cyclase, |
| | complete cds |
| 701521645 | Rat aldehyde dehydrogenase mRNA, complete cds |
| 701562834 | Rat thymosin β-10 gene, complete cds |

By selecting for genes that distinguish gemfibrozil from other fibrates, we have essentially subtracted the “fibrate activity” from the signature. The signature remaining indicates an additional activity, which in this case happens to correlate to a known side effect: gemfibrozil is known to induce an increase in LDL (low density lipoprotein) levels in hypertriglyceridemic patients.

Various computer systems, typically comprising one or more microprocessors, can be used to store, retrieve, and analyze information obtained according to the methods of the invention. The computer systems can be as simple as a stand-alone computer having a form of data storage (i.e., a computer-readable medium, such as, for example, a floppy disk, a hard drive, removable disk storage such as a ZIP® drive, optical medium such as CD-ROM and DVD, magnetic tape, solid-state memory, magnetic bubble memory, and the like). Alternatively, the computer system can include a network comprising two or more computers linked together, for example through a network server. The network can comprise an intranet, an Internet connection, or both. In one embodiment of the invention, a stand-alone computer system is provided with a computer-readable medium containing a Group Signature database thereon, said Group Signature database comprising one or more Group Signature records. The computer system preferably further comprises a processor and software that enables the system to compare gene expression and/or bioassay data from an experiment with the contents of the Group Signature database. In another embodiment of the invention, a computer is provided with a computer-readable medium containing a Group Signature database thereon (a database server), and a network connection over which other computers can connect (user systems). Preferably, the user systems are provided with processors and software for receiving and storing gene expression and/or bioassay data from one or more experiments, and for formulating database queries for transmission over the network and execution on either the database server or on the user system. The computer system can further be linked to additional databases such as Genbank and DrugMatrix (Iconix Pharmaceuticals, Inc., Mountain View, Calif.).

### EXAMPLE

The following examples are provided as a guide for the practitioner of ordinary skill in the art. Nothing in the examples is intended to limit the claimed invention. Unless otherwise specified, all reagents are used in accordance with the manufacturer's recommendations.

### Example 1

### Fibrate Drug Signature

(A) Data Collection

Sprague-Dawley Crl:CD(SD) BR strain (VAF plus) rats aged 4-6 weeks were fed a standard rodent diet and allowed tap water ad libitum. Animal procedures were carried out at Sequani Ltd. (Ledbury, Herefordshire, England).

All compounds were administered to groups of two male and two female rats for each dose and time. Estradiol benzoate, bisphenol A (“BPA”) and octylphenol (“OP”) were administered subcutaneously in arrachis oil; clofibrate, fenofibrate, gemfibrozil and bis(2-ethyl-hexyl)phthalate (“DEHP”) were administered by oral gavage in 1% NaCMC. The doses used were the maximum tolerated dose (MTD), 70% MTD, 50% MTD, and 10% MTD for each compound. All MTDs were determined from the literature or based on experience. The MTDs used were: estradiol benzoate=2 mg/kg; BPA=150 mg/kg; OP=450 mg/kg; clofibrate=250 mg/kg; fenofibrate=1,000 mg/kg; gemfibrozil=300 mg/kg; DEHP=1,000 mg/kg. Tissues were harvested at 3, 24, or 72 hours after the initial dose. For the 3 and 24 hour time points, animals were dosed at time 0 and euthanized at 3 hours and 24 hours, respectively. For the 72 hour time point, animals were dosed at 0, 24, and 48 hours, then euthanized at 72 hours. Tissues were collected and frozen on dry ice prior to storage at −80° C.

Homogenization of liver tissue, mRNA extraction, and probe labeling were performed as described by H. Yue et al., Nuc Acids Res (2001) 29(8):E41-1, incorporated herein by reference. Each sample was hybridized to duplicate Rat Toxicology LifeArrays (Incyte Genomics, Palo Alto, Calif.) as described by J. L. DeRisi et al., Science (1997) 278(5338):680-86, incorporated herein by reference. The control mRNA was derived from a pool of livers obtained from age- and strain-matched untreated animals (40 male and 40 female). All 680 microarrays were analyzed simultaneously after mean total signal intensity normalization across both channels using GEM Tools®. Gene regulation was expressed as log₂ of the normalized ratios. Missing values were replaced with log₂ ratios=0.

The 200 genes displaying the greatest variability was determined by the standard deviation of the ratios of a clone across all 680 experiments (listed in Table 1 below). These genes were selected as variables for principal component analysis (PCA) using Spotfire™ DecisionSite™ 6.3. The most important genes were identified by sorting their eigenvalue for each PCA dimension.

TABLE 1
Genes with highest variability to fibrates

| Accession # | Clone ID | Name |
|---|---|---|
| K03249 | 700935113 | Rat peroxisomal enoyl-CoA: hydrotase-3-hydroxyacyl-CoA |
| | | bifunctional enzyme mRNA, complete cds |
| J00738 | 700295656 | Rat submaxillary gland alpha-2μ globulin mRNA, complete |
| | | cds |
| U41394 | 700523053 | Mouse X inactivation transcript (Xist) gene, cosmid MB4- |
| | | 14A, fragment 1 |
| M97167 | 700812060 | Mouse X (inactive)-specific transcript (Xist) 5′ repeat region, |
| | | partial mRNA sequence |
| M14972 | 701444552 | Rat cytochrome P450-LA-omega (lauric acid omega- |
| | | hydroxylase) mRNA, complete cds |
| X07259 | 701507855 | Rat mRNA for cytochrome P452 |
| AF037072 | 700820751 | Rat carbonic anhydrase III (CA3) mRNA, complete cds |
| CAC19029 | 700607235 | liver regeneration-related protein 1 |
| V01216 | 701192802 | Rat α1-acid glycoprotein (AGP) mRNA, complete cds |
| M31363 | 700610331 | Rat hydroxysteroid sulfotransferase mRNA, complete cds |
| M13524 | 700610669 | Mouse serum amyloid A pseudogene (psi-SAA) |
| M29301 | 701879735 | Rat senescence marker protein 2A gene, exons 1 and 2 |
| X79991 | 701257404 | Rat CYP3 mRNA |
| X67156 | 700270866 | Rat mRNA for (S)-2-hydroxy acid oxidase |
| U33500 | 700301147 | Rat retinol dehydrogenase type II mRNA, complete cds |
| AB017446 | 701430253 | Rat mRNA for organic anion transporter 3, complete cds |
| M37828 | 700296634 | Rat cytochrome P450 mRNA, complete cds |
| M14972 | 701466373 | Rat cytochrome P450-LA-omega (lauric acid omega- |
| | | hydroxylase) mRNA, complete cds |
| U31287 | 701727292 | Rat α2u globulin mRNA, complete cds |
| U41394 | 701441211 | Mouse X inactivation transcript (Xist) gene, cosmid MB4- |
| | | 14A, fragment 1 |
| M33936 | 701196893 | Rat Cyp4a locus, encoding cytochrome P450 (IVA3) mRNA, |
| | | complete cds |
| X61184 | 700481210 | Rat mRNA for mitochondrial 3-2trans-enoyl-CoA isomerase |
| M37828 | 700296865 | Rat cytochrome P450 mRNA, complete cds |
| AJ224120 | 701512110 | Rat peroxisomal membrane protein Pmp26p (Peroxin-11) |
| X96721 | 700606819 | Rat mRNA for P450IIIA23 protein |
| 0 | 700305024 | Incyte EST |
| M27883 | 701191029 | Rat pancreatic secretory trypsin inhibitor type II (PSTI-II) |
| | | mRNA, complete cds |
| AB010428 | 701466951 | Rat mRNA for acyl-CoA hydrolase, complete cds |
| Y10420 | 700252601 | Rat gene encoding 11β-hydroxysteroid dehydrogenase 1 |
| U08976 | 700247611 | Rat Wistar peroxisomal enoyl hydratase-like protein (PXEL) |
| | | mRNA, complete cds |
| 0 | 701461734 | Incyte EST |
| M11794 | 700501633 | Rat metallothionein-2 and metallothionein-1 genes, complete |
| | | cds |
| BAA91273 | 701428215 | Unnamed protein product |
| BAA91069 | 700148731 | Unnamed protein product |
| X13295 | 700483986 | Rat mRNA for α2u globulin-related protein. |
| M11794 | 700176945 | Rat metallothionein-2 and metallothionein-1 genes, complete |
| | | cds |
| AB017446 | 701263974 | Rat mRNA for organic anion transporter 3, complete cds |
| U26033 | 701531239 | Rat carnitine octanoyltransferase mRNA, complete cds |
| AF182168 | 700482728 | Rat aldose-reductase-like protein MVDP/AKR1-B7 mRNA, |
| | | complete cds |
| U46118 | 700513352 | Rat cytochrome P450 3A9 mRNA, complete cds |
| J02752 | 701646795 | Rat acyl-CoA oxidase mRNA, complete cds |
| AAC36536 | 700532842 | Unknown |
| K03243 | 700594016 | Rat phosphoenolpyruvate carboxykinase (GTP) gene exons 1-3 |
| K03249 | 701469162 | Rat peroxisomal enoyl-CoA: hydrotase-3-hydroxyacyl-CoA |
| | | bifunctional enzyme mRNA, complete cds |
| 0 | 700503535 | Incyte EST |
| X16359 | 700364565 | Rat mRNA for SPI-3 serine protease inhibitor |
| J03621 | 701193790 | Rat mitochondrial succinyl-CoA synthetase alpha subunit |
| | | (cytoplasmic precursor) mRNA, complete cds |
| J05035 | 700588986 | Rat steroid 5α-reductase mRNA, complete cds |
| U04204 | 700182878 | Mouse BALB/c aldose reductase-related protein mRNA, |
| | | complete cds |
| X12595 | 700610052 | Rat gene for cytochrome P450 f |
| M11794 | 700814596 | Rat metallothionein-2 and metallothionein-1 genes, complete |
| | | cds |
| J03621 | 701195413 | Rat mitochondrial succinyl-CoA synthetase alpha subunit |
| | | (cytoplasmic precursor) mRNA, complete cds |
| J02585 | 700330140 | Rat liver stearyl-CoA desaturase mRNA, complete cds |
| AF180801 | 701606788 | Mouse peroxisomal long chain acyl-CoA thioesterase Ib |
| | | (Ptelb) gene, exon 3 and complete cds |
| CAB08313 | 700228072 | hypothetical protein Rv3224 |
| M13508 | 700287180 | Rat apolipoprotein A-IV gene, complete cds |
| AAD34081 | 701258991 | CGI-86 protein |
| CAB08313 | 701826047 | hypothetical protein Rv3224 |
| J00732 | 700505778 | Rat liver fatty acid binding protein (FABP) mRNA. |
| D13921 | 700370576 | Rat mitochondrial acetoacetyl-CoA thiolase mRNA, complete |
| | | cds |
| X91234 | 700606955 | Rat mRNA for 17β hydroxysteroid dehydrogenase type 2 |
| AAF65568 | 700607496 | Thymus-expressed novel gene-3 protein |
| 0 | 700543841 | Incyte EST |
| AF060490 | 700245238 | Mouse TLS-associated protein TASR-2 mRNA, complete cds |
| 0 | 700480077 | Incyte EST |
| L22339 | 701259952 | Rat N-hydroxy-2-acetylaminofluorene (ST1C1) mRNA, |
| | | complete cds |
| AB030184 | 701342654 | Mouse mRNA, complete cds, clone: 1-44 |
| K01933 | 700607255 | Rat haptoglobin mRNA, partial α-, complete β-subunit and 3′ |
| | | flank. |
| X52625 | 700147478 | Rat mRNA for cytosolic 3-hydroxy 3-methylglutaryl CoA |
| | | synthase (EC 4.1.3.5) |
| M11842 | 700508056 | Rat ornithine aminotransferase mRNA, complete cds |
| AAF52911 | 700302116 | CG4995 gene product |
| BAA91273 | 701880740 | unnamed protein product |
| AF198441 | 700483163 | Rat urinary protein 2 precursor, mRNA, complete cds |
| D78592 | 700937302 | Rat mRNA for glucose-6-phosphatase catalytic subunit, |
| | | complete cds |
| D90038 | 701427356 | Rat liver 70-kDa peroxisomal membrane protein (PMP70) |
| | | mRNA |
| D28560 | 700860387 | Rat mRNA for phosphodiesterase I |
| M33648 | 700199767 | Rat mitochondrial 3-hydroxy-3-methylglutaryl-CoA synthase |
| | | mRNA, complete cds |
| AF121351 | 701878550 | Mouse chromosome X clone BAC B22804, complete sequence |
| M23995 | 701234495 | Rat aldehyde dehydrogenase mRNA, complete cds |
| 0 | 700638749 | Incyte EST |
| AJ238392 | 701197528 | Rat mRNA for Sulfotransferase K2 |
| X05341 | 700147217 | Rat mRNA for 3-oxoacyl-CoA thiolase |
| X96553 | 701519057 | Rat mRNA for hepatocyte nuclear factor 6α. |
| X07365 | 700181385 | Rat mRNA for glutathione peroxidase |
| 0 | 701882512 | Incyte EST |
| M38179 | 700268926 | Rat 3β-hydroxysteroid dehydrogenase/Δ-5-Δ-4 isomerase type |
| | | II (3-β-HSD) mRNA, complete cds |
| 0 | 701702593 | Incyte EST |
| U87602 | 700610575 | Rat L1 retrotransposon mlvi2-rn14, 5′UTR and putative RNA |
| | | binding protein 1 gene, partial cds |
| AJ132098 | 700933512 | Mouse mRNA for Vanin-1 |
| K00034 | 700531210 | rat u2 small nuclear RNA gene and flanks |
| X65083 | 700228203 | Rat mRNA for cytosolic epoxide hydrolase |
| X85983 | 700435732 | Mouse mRNA for carnitine acetyltransferase |
| M62642 | 700502986 | Rat (clone pRHx1) hemopexin mRNA, complete cds |
| J00734 | 701431517 | rat fibrinogen γ chain-a mRNA |
| X86561 | 700503328 | Rat gene for α-fibrinogen. |
| AB009686 | 701244533 | Rat CYP8B mRNA for sterol 12α-hydroxylase P450, complete |
| | | cds |
| AAG36780 | 700607052 | inorganic pyrophosphatase |
| 0 | 701436464 | Incyte EST |
| AF169157 | 700938509 | Mouse L-CaBP2 (Cabp2) mRNA, complete cds |
| U05675 | 700606793 | Rat Sprague-Dawley fibrinogen B β chain mRNA, complete |
| | | cds |
| M86758 | 701256292 | Rat estrogen sulfotransferase mRNA, complete cds |
| U26033 | 701227715 | Rat carnitine octanoyltransferase mRNA, complete cds |
| AAA60043 | 700309689 | endothelial cell growth factor |
| AF044574 | 701030993 | Rat putative peroxisomal 2,4-dienoyl-CoA reductase (DCR- |
| | | AKL) mRNA, complete cds |
| X13415 | 700290539 | Rat fatty acid synthase mRNA, complete cds |
| 0 | 700484751 | Incyte EST |
| D00569 | 700628567 | Rat mRNA for 2,4-dienoyl-CoA reductase precursor, complete |
| | | cds |
| AC020967 | 700483248 | Mouse chromosome 18 clone RP23-161O8, complete |
| | | sequence |
| 0 | 701512411 | Incyte EST |
| AF034577 | 700187344 | Rat pyruvate dehydrogenase kinase isoenzyme 4 (PDK4) |
| | | mRNA, complete cds |
| CAA72272 | 700528633 | Phosphoenolpyruvate carboxykinase (GTP) |
| AF001896 | 700509013 | Rat aldehyde dehydrogenase mRNA, complete cds |
| Y12517 | 700935253 | Rat mRNA for mitochondrial isoform of cytochrome b5 |
| M58634 | 701186676 | Rat IGF binding protein-1 (rIGFBP-1) mRNA, complete cds |
| AAD45920 | 701336191 | angiopoietin-related protein 3 |
| AF038870 | 700607442 | Rat betaine homocysteine methyltransferase (BHMT) mRNA, |
| | | complete cds |
| M23721 | 700198507 | Rat carboxypeptidase (CA2) gene, exon 11 |
| Y11283 | 700305148 | Rat mRNA for plasma protein. |
| X53477 | 700304380 | Rat p450Md mRNA for cytochrome P450 |
| U15566 | 701560684 | Mouse Tbx2 mRNA, complete cds |
| D90038 | 700288719 | Rat liver 70-kDa peroxisomal membrane protein (PMP70) |
| | | mRNA |
| AF202115 | 701463794 | Rat GPI-anchored ceruloplasmin mRNA, complete cds |
| S78221 | 700606373 | nuclear protein TIF1 isoform (Mouse, mRNA, 4053 nt) |
| #N/A | 700138684 | Mouse L-CaBP2 (Cabp2) mRNA, complete cds |
| X53725 | 700329424 | Rat MASH-1 mRNA expressed in neuronal precursor cells |
| | | (mammalian achaete-scute homologue) |
| U40397 | 700938882 | Mouse serum amyloid A-4 protein (Saa4) gene, complete cds |
| M23995 | 701521645 | Rat aldehyde dehydrogenase mRNA, complete cds |
| 0 | 700931483 | Incyte EST |
| D28566 | 701192728 | Hamster mRNA for carboxylesterase precursor, complete cds |
| M13590 | 700147294 | Rat glutathione S-transferase Yb2 subunit mRNA, 3′ end |
| AAF09483 | 701644022 | E2IG4 |
| 0 | 700515449 | Incyte EST |
| AB002558 | 700626043 | Rat mRNA for glycerol 3-phosphate dehydrogenase, complete |
| | | cds |
| AJ302031 | 700503842 | Rat liver regeneration-related protein 1 mRNA, complete cds |
| D16479 | 700397284 | Rat mRNA for mitochondrial long-chain 3-ketoacyl-CoA |
| | | thiolase β-subunit of mitochondrial trifunctional protein, |
| | | complete cds |
| AE000664 | 700503071 | Mouse T-cell receptor α locus BAC clone MBAC519 from |
| | | 14D1-D2, complete sequence |
| AB010428 | 700146486 | Rat mRNA for acyl-CoA hydrolase, complete cds |
| AF117887 | 700245634 | Mouse protein arginine methyltransferase (Carm1) mRNA, |
| | | complete cds |
| U43285 | 700368469 | Mouse selenophosphate synthetase 2 mRNA, complete cds |
| U42719 | 701438090 | Rat C4 complement protein mRNA, partial cds |
| AAA65642 | 700502628 | apolipoprotein F |
| S83247 | 700233325 | DA11 = 15.2 kDa fatty acid binding protein/FABP/C-FAPB |
| | | homolog (rats, Sprague-Dawley, sciatic nerve traumatized, |
| | | dorsal root ganglia, mRNA Partial, 695 nt) |
| AAA36986 | 700608519 | glutathione S-transferase subunit pi |
| M59189 | 701436793 | Rat cholesterol 7α-hydroxylase gene, exon 6 |
| 0 | 701644979 | Incyte EST |
| AF116897 | 701193378 | Mouse mahogany protein mRNA, complete cds |
| M80427 | 700303313 | Syrian golden hamster androgen-dependent expressed protein |
| | | mRNA, complete cds |
| M14201 | 700487123 | Rat 11-Kd diazepam binding inhibitor (DBI), partial cds |
| D88250 | 700372447 | Rat mRNA for serine protease, complete cds |
| #N/A | 700063031 | Rat VL30 element mRNA |
| D37920 | 700491942 | Rat mRNA for squalene epoxidase, complete cds |
| U61266 | 700522707 | Rat Rho-associated kinase β mRNA, complete cds |
| U02553 | 700187524 | Rat protein tyrosine phosphatase mRNA, complete cds |
| AF062389 | 700304757 | Rat kidney-specific protein (KS) mRNA, complete cds |
| D50559 | 700513027 | Rat mRNA for RANP-1, complete cds |
| K02422 | 701193624 | Rat cytochrome P450d methylcholanthrene-inducible gene, |
| | | complete cds |
| X05684 | 701559151 | Rat L-PK gene for L-type pyruvate kinase |
| M11709 | 701345507 | Rat L-type pyruvate kinase mRNA, complete cds |
| M20131 | 700502447 | Rat cytochrome P450IIE1 gene, complete cds |
| X07266 | 700492544 | Rat mRNA for gene 33 polypeptide |
| V01222 | 701431070 | Messenger RNA for rat preproalbumin |
| J04632 | 700484528 | Mouse glutathione S-transferase class μ (GST1-1) mRNA, |
| | | complete cds |
| J05430 | 701487679 | Rat cholesterol 7α-hydroxylase (CYP7) mRNA, complete cds |
| M77003 | 700331551 | Mouse glycerol-3-phosphate acyltransferase mRNA, complete |
| | | cds |
| J03734 | 701194460 | Rat Kupffer cell receptor mRNA, complete cds |
| Z50051 | 700610324 | R. norvegicus mRNA for Bovine C4BP α-chain protein |
| 0 | 701437076 | Incyte EST |
| D90005 | 701430626 | Rat endogenous retroviral sequence, 5′ and 3′ LTR |
| BAB14526 | 701826510 | oxidoreductase UCPA |
| U38419 | 700609878 | Rat dopa/tyrosine sulfotransferase mRNA, complete cds |
| AF110477 | 701482962 | Rat liver aldehyde oxidase female form (AOX1) mRNA, |
| | | complete cds |
| S74802 | 700178702 | Rat beta-globin gene, exons 1-3 |
| M34561 | 700146495 | Rat 70 kd heat-shock-like protein mRNA, complete cds |
| 0 | 701440048 | Incyte EST |
| X05341 | 700228787 | Rat mRNA for 3-oxoacyl-CoA thiolase |
| AF172276 | 701649184 | Mouse aldehyde oxidase homolog-1 (Aoh1) mRNA, complete |
| | | cds |
| AF044574 | 701246587 | Rat putative peroxisomal 2,4-dienoyl-CoA reductase (DCR- |
| | | AKL) mRNA, complete cds |
| D90109 | 700527892 | Rat mRNA for long-chain acyl-CoA synthetase (EC 6.2.1.3) |
| #N/A | 700137495 | Rat pcRC201 mRNA for pre-pro-complement C3 |
| X03430 | 700484501 | Rat mRNA for L-type pyruvate kinase |
| AF216873 | 700183232 | Mouse acetyl-CoA synthetase mRNA, complete cds |
| M58404 | 701562834 | Rat thymosin β-10 gene, complete cds |
| M12516 | 700304405 | Rat NADPH-cytochrome P450 reductase mRNA, complete cds |
| 0 | 700501620 | Incyte EST |
| K03252 | 700481289 | Rat prealbumin (transthyretin) mRNA, complete cds |
| X52984 | 700609873 | Rat mRNA for alpha(1)-inhibitor 3, variant I |
| 0 | 700930555 | Incyte EST |
| 0 | 700328880 | Incyte EST |
| Z32548 | 701430793 | Mouse TRGC78 DNA 414 bp |
| 0 | 701518575 | Incyte EST |
| BAA34502 | 700180621 | KIAA0782 protein |
| U49071 | 700304375 | Rat complement component C9 precursor mRNA, partial cds |
| AB012276 | 700528176 | Mouse mRNA for ATFx, partial cds |
| AB010632 | 700480022 | Rat mRNA for carboxylesterase precursor, complete cds |
| 0 | 700483266 | Incyte EST |
| J02861 | 701193056 | Rat polymorphic, male-specific cytochrome P450g mRNA, |
| | | complete cds |
| AF200357 | 701258381 | Mouse pantothenate kinase 1β (panK1β) mRNA, complete |
| | | cds. |
| D45252 | 701228305 | Rat mRNA for 2,3-oxidosqualene: lanosterol cyclase, complete |
| | | cds |
| D17370 | 700307241 | Rat mRNA for cystathionine gamma-lyase, complete cds |
| M17083 | 700293050 | Rat major alpha-globin mRNA, complete cds |

Molecular pharmacology assays were performed on all compounds in 130 different assays selected from the MDS-Pharma Services catalog. The panel of assays was chosen to include important sites of drug action and drug toxicity. Those compounds that exhibited a fractional inhibition of ≧50% at 30 μM in a preliminary duplicate test were further studied using an eight-point triplicate concentration titration at ½-log intervals from 30 μM, to determine an IC₅₀ value.

(B) Analysis

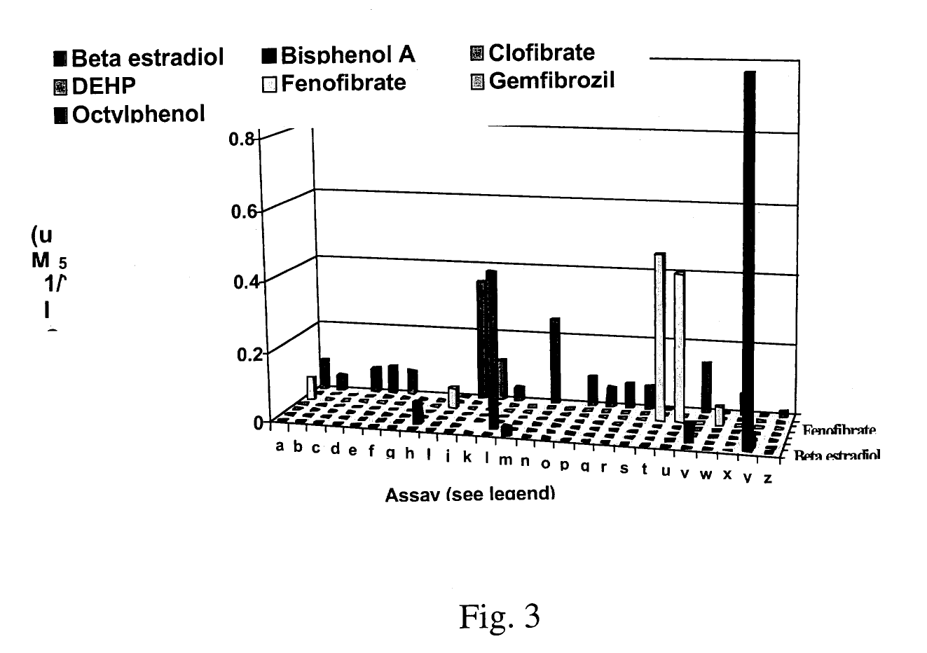

FIG. 3 sets forth the results of the bioassay experiments. Compound measurements that resulted in <50% inhibition were binned as 0. Gemfibrozil, clofibrate and DEHP demonstrated no activity in the 123 assays completed. In contrast, OP interacted in 16 of the 123 assays conducted. Fenofibrate interacted weakly with the estrogen receptor and the site-2 sodium channel, and potently with 5HT2a and 5HT2c with Kds of about 600 nM. This finding suggests other novel mechanisms of action and applications for fibrates that merit further investigation.

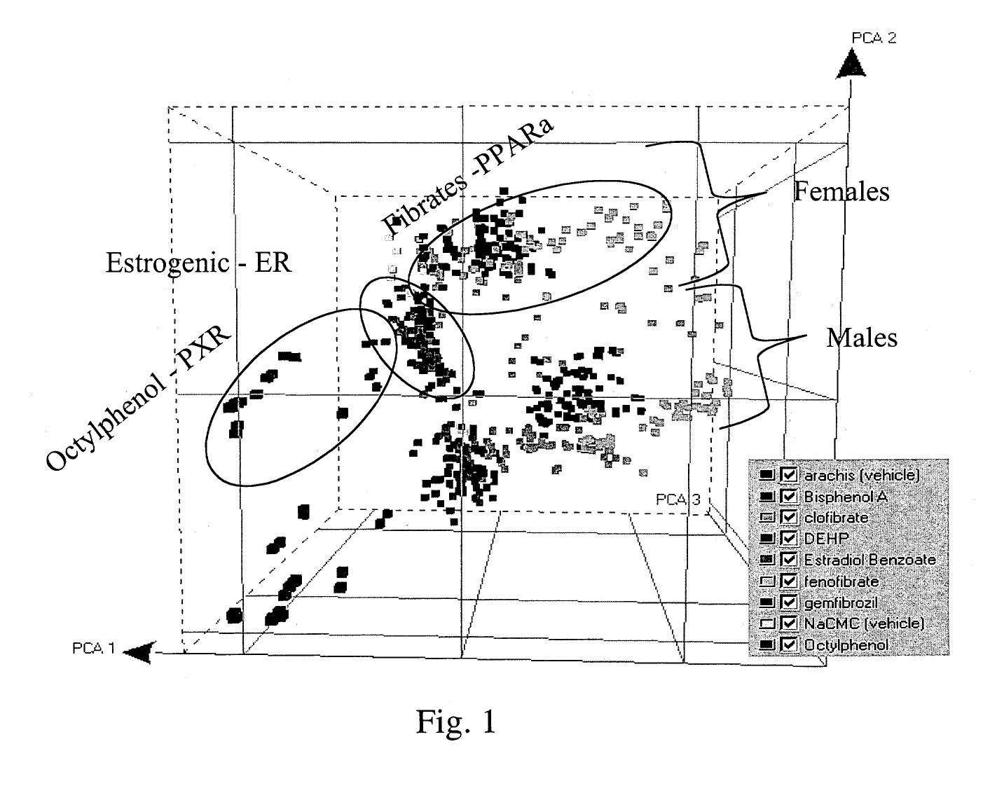

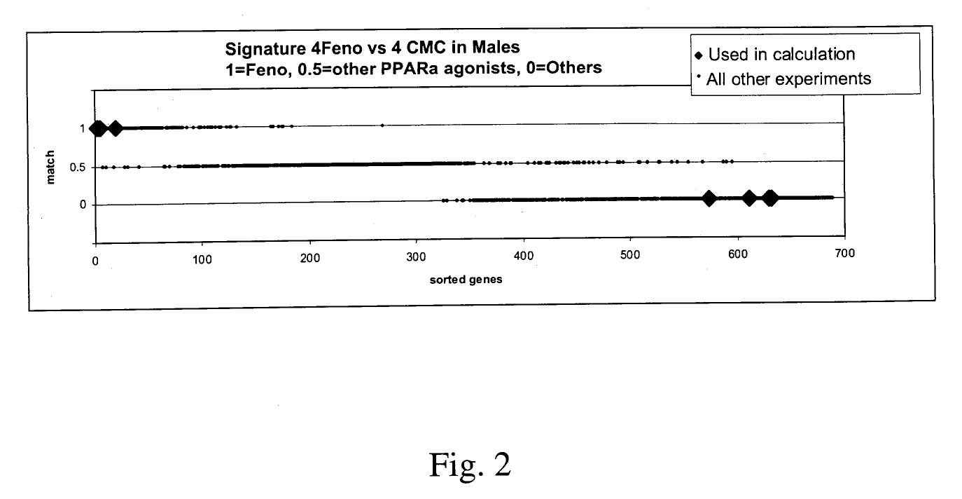

The 200 genes displaying the greatest difference in expression level between experimental and control groups were selected for principal component analysis (PCA). Compounds (rather than genes) were sorted and clustered by PCA, and displayed in a 3D depiction as shown in FIG. 1. The results indicate that the expression patterns cluster into several distinct groups. Fibrates and other peroxisomal proliferator compounds such as DEHP cluster in one group, while estradiol benzoate and BPA (both pure estrogen receptor agonists) and the vehicle controls group into a second group. OP, a weak estrogen receptor (“ER”) agonist that also has activity on the PXR, separates from the other compounds in a unique position. Each of the groups was further split according to gender of the test animal.

The three PCA components were then examined to determine which genes contributed to each component. The results are set forth in Table 4 below, which lists the genes that contribute most to the first principal component, along with their contribution to each principal component. The first PCA component is dominated by the effect of peroxisomal proliferator PPARα agonists (the fibrates and DEHP), and is associated primarily with fatty acid beta oxidation gene expression. The effect of gender on the expression of certain genes, particularly 4-12 X-chromosome specific transcripts and certain sex steroid metabolism genes dominate the second principal component. The third component was dominated by the effect of OP (PXR/ER mixed agonist), and is associated with extracellular and blood protein genes that might be indicative of stress responses. The ER-selective agonists (estradiol benzoate and BPA) and the vehicles were unresolved.

TABLE 4
Genes by Principal Component Contribution sorted by PC(1)
Eigenvalues (Top X genes shown)

| Clone ID | Gene | PC(1) | PC(2) | PC(3) |
|---|---|---|---|---|
| 700935113 | Rat peroxisomal enoyl-CoA: hydrotase-3- | 0.26 | −7.00E−2 | 5.80E−2 |
| | hydroxyacyl-CoA bifunctional enzyme | | | |
| | mRNA, complete cds | | | |
| 701466373 | Rat cytochrome P450-LA-omega (lauric acid | 0.216 | −5.00E−2 | 0.101 |
| | omega-hydroxylase) mRNA, complete cds | | | |
| 701507855 | Rat mRNA for cytochrome P452 | 0.204 | −4.40E−2 | 8.80E−2 |
| 700296865 | Rat cytochrome P450 mRNA, complete cds | 0.182 | −1.40E−2 | 8.60E−2 |
| 701444552 | Rat cytochrome P450-LA-omega (lauric acid | 0.182 | −3.80E−2 | 8.90E−2 |
| | omega-hydroxylase) mRNA, complete cds (2) | | | |
| 700296634 | Rat cytochrome P450 mRNA, complete cds (2) | 0.171 | −1.40E−2 | 9.50E−2 |
| 700247611 | Rat Wistar peroxisomal enoyl hydratase-like | 0.169 | −3.20E−2 | 3.40E−2 |
| | protein (PXEL) mRNA, complete cds | | | |
| 701196893 | Rat Cyp4a locus, encoding cytochrome P450 | 0.168 | 1.10E−2 | 9.40E−2 |
| | (IVA3) mRNA, complete cds | | | |
| 700481210 | Rat mRNA for mitochondrial 3-2-trans-enoyl- | 0.165 | −5.80E−2 | 2.50E−2 |
| | CoA isomerase | | | |
| 701512110 | Rat peroxisomal membrane protein Pmp26p | 0.16 | −4.40E−2 | 7.60E−2 |
| | (Peroxin-11) | | | |
| 700146486 | Rat mRNA for acyl-CoA hydrolase, complete | 0.159 | −6.90E−2 | 1.00E−1 |
| | cds | | | |
| 701646795 | Rat acyl-CoA oxidase mRNA, complete cds | 0.151 | −3.40E−2 | 7.40E−2 |
| 701469162 | Rat peroxisomal enoyl-CoA: hydrotase-3- | 0.147 | −3.20E−2 | 5.80E−2 |
| | hydroxyacyl-CoA bifunctional enzyme | | | |
| | mRNA, complete cds (2) | | | |
| 701531239 | Rat carnitine octanoyltransferase mRNA, | 0.143 | −3.40E−2 | 5.60E−3 |
| | complete cds | | | |
| 700295656 | Rat submaxillary gland alpha-2μ globulin | 0.137 | 0.139 | −8.40E−2 |
| | mRNA, complete cds | | | |
| 700370576 | Rat mitochondrial acetoacetyl-CoA thiolase | 0.123 | 1.10E−2 | −4.2E−2 |
| | mRNA, complete cds | | | |
| 701826047 | hypothetical protein Rv3224 | 0.121 | −3.20E−2 | 3.30E−2 |
| 701880740 | Unnamed protein product | 0.119 | −2.40E−2 | −4.40E−3 |

The separation of the PPARα agonists in one component, estradiol and BPA in another, and OP in a third correlates with the activities of these compounds on several receptors expressed in liver. DEHP and the fibrates potently stimulate PPARα, and their toxicity in liver requires the presence of PPARα (J. C. Corton et al., Ann. Rev. Pharmacol. Toxicol. (2000) 40:491-518; J. M. Ward et al., Toxicol. Pathol. (1998) 26(2):240-46; S. A. Kliewer et al., Science (1999) 284(5415):757-60). These activities correlate with the clustering of the PPARα agonists in the PCA. Estradiol stimulates the estrogen receptor with an ED₅₀ near 10⁻¹¹ M (H. Masuyama et al., Mol. Endocrinol. (2000) 14(3):421-28), while BPA, DEHP and nonylphenol (a homolog of OP) stimulate ER with EC₅₀s of about 1 μM. DEHP and nonylphenol stimulate PXR receptors with an EC₅₀ of approximately 0.5 μM, while estradiol and BPA are completely inactive on PXR (H. Masuyama et al., supra). ER-active compounds (estradiol and BPA) clustered with the vehicle controls, perhaps because liver shows weak estrogenic response. Because DEHP, which is active on PXR, did not induce the same genes as OP, the distinction may arise from activity on one or multiple other receptors. The potential activity of OP on other receptors is supported by its promiscuity in the molecular pharmacology assays above (see also H. Masuyama et al., supra).

To better understand which genes drive the discrimination of PPARα agonists (“PP”) from the ER and ER/PXR compounds (“Non”), the data were also analyzed using the discrimination metric developed by T. R. Golub et al., Science (1999) 286(5439):531-37. These calculations identified a number of genes that uniquely discriminate the PP set from the Non set. Of the top 100 most discriminating genes for PP, 35 were readily identified as belonging to the fatty acid beta oxidation (FABO) pathway, and 25 were novel genes. We suggest that some or all of these novel genes are also member of the FABO pathway, previously unrecognized. Table 5 below shows the distinctiveness value for the top 25 genes identified as highly distinctive for fibrates, comparing fenofibrate vs. vehicle (in males), clofibrate vs. vehicle (in males), and (for comparison) the non-fibrate octylphenol vs. vehicle (in males). In this table, negative values indicate upregulation and positive values indicate down-regulation. It is clear from the table that fenofibrate and clofibrate are closely correlated, differing mainly in the degree of upregulation, and that both are essentially non-correlated with octylphenol. This demonstrates that the method of the invention is capable of distinguishing different biological activities based on gene expression patterns, and that it is capable of identifying the relevant genes. Further, it demonstrates that the method of the invention is capable of finding genes having previously unknown activity (for example, the “Unnamed protein product”), and grouping them with genes of known activity.

TABLE 5
Distinctiveness

| Clone ID | Gene | Fenofibrate | Clofibrate | Octylphenol |
|---|---|---|---|---|
| 701507855 | Rat mRNA for cytochrome P452 | −32.35 | −11.54 | 1.43 |
| 700296865 | Rat cytochrome P450 mRNA, | −25.96 | −17.07 | 0.69 |
| | complete cds | | | |
| 701466373 | Rat cytochrome P450-LA-omega | −24.45 | −23.12 | 1.09 |
| | (lauric acid omega-hydroxylase) | | | |
| | mRNA, complete cds | | | |
| 701197528 | Rat mRNA for Sulfotransferase K2 | −24.19 | −29.46 | 1.16 |
| 701444552 | Rat cytochrome P450-LA-omega | −22.11 | −15.09 | 0.84 |
| | (lauric acid omega-hydroxylase) | | | |
| | mRNA, complete cds | | | |
| 701196893 | Rat Cyp4a locus, encoding | −21.68 | −15.66 | −0.81 |
| | cytochrome P450 (IVA3) mRNA, | | | |
| | complete cds | | | |
| 700296634 | Rat cytochrome P450 mRNA, | −19.23 | −15.64 | 0.69 |
| | complete cds | | | |
| 700481210 | Rat mRNA for mitochondrial 3-2- | −19.18 | −11.57 | 2.16 |
| | trans-enoyl-CoA isomerase | | | |
| 701531239 | Rat carnitine octanoyltransferase | −18.56 | −7.50 | 3.56 |
| | mRNA, complete cds | | | |
| 701880740 | Unnamed protein product | −17.88 | −1.20 | 3.42 |
| 700247611 | Rat Wistar peroxisomal enoyl | −16.48 | −10.02 | 8.18 |
| | hydratase-like protein (PXEL) | | | |
| | mRNA, complete cds | | | |
| 700397284 | Rat mRNA for mitochondrial long- | −14.54 | −4.48 | 1.46 |
| | chain 3-ketoacyl-CoA thiolase β- | | | |
| | subunit of mitochondrial | | | |
| | trifunctional protein, complete cds | | | |
| 700505778 | Rat liver fatty acid binding protein | −14.06 | −0.23 | 3.92 |
| | (FABP) mRNA. | | | |
| 700187344 | Rat pyruvate dehydrogenase kinase | −13.16 | −16.42 | −0.19 |
| | isoenzyme 4 (PDK4) mRNA, | | | |
| | complete cds | | | |
| 700935253 | Rat mRNA for mitochondrial | −13.15 | −4.98 | 1.37 |
| | isoform of cytochrome b5 | | | |
| 701826047 | hypothetical protein Rv3224 | −12.66 | −5.56 | 1.36 |
| 701512411 | Incyte EST | -11.10 | −6.28 | 0.96 |
| 700935113 | Rat peroxisomal enoyl-CoA: | −10.93 | −4.82 | 1.40 |
| | hydrotase-3-hydroxyacyl-CoA | | | |
| | bifunctional enzyme mRNA, | | | |
| | complete cds | | | |
| 701512110 | Rat peroxisomal membrane protein | −10.83 | −8.16 | 1.31 |
| | Pmp26p (Peroxin-11) | | | |
| 700146486 | Rat mRNA for acyl-CoA hydrolase, | −10.45 | −2.20 | −2.66 |
| | complete cds | | | |
| 701646795 | Rat acyl-CoA oxidase mRNA, | −10.07 | −8.02 | 0.81 |
| | complete cds | | | |
| 701466951 | Rat mRNA for acyl-CoA hydrolase, | −9.93 | −7.60 | −1.06 |
| | complete cds | | | |
| 700628567 | Rat mRNA for 2,4-dienoyl-CoA | −8.98 | −2.16 | 2.72 |
| | reductase precursor, complete cds | | | |
| 700199767 | Rat mitochondrial 3-hydroxy-3- | −8.72 | −9.90 | 1.97 |
| | methylglutaryl-CoA synthase | | | |
| | mRNA, complete cds | | | |
| 701469162 | Rat peroxisomal enoyl-CoA: | −7.82 | −5.72 | 1.22 |
| | hydrotase-3-hydroxyacyl-CoA | | | |
| | bifunctional enzyme mRNA, | | | |
| | complete cds | | | |
| 701606788 | Mouse peroxisomal long chain acyl- | −7.74 | −6.91 | 1.91 |
| | CoA thioesterase Ib (Ptelb) gene, | | | |
| | exon 3 and complete cds | | | |

PCA and discrimination calculations identified a strongly overlapping set of genes: 14 of the top 15 genes identified by PCA were also identified in the top 100 most distinctive genes. The discrimination of PPARα agonists from the other drugs by two methods provides a cross-validation of the results, suggesting that the FABO pathway is a defining effect of the PPAR-agonist drugs.

A group signature was derived by identifying the top 20 genes most capable of discriminating PPARα compounds from non-PPARα compounds, and from this group selecting the genes that responded most consistently for all members of the PPARα-agonist group of compounds. For fibrate signature (determined on fenofibrate versus vehicle) the top ten genes upregulated genes work as well as the top 20. In essence, the selection of only a few genes from the group signature was sufficient to distinguish the common activity of the PPARα compounds from the activity of other compounds. Inclusion of additional genes selected from the group signature increases the degree of confidence. For example, a signature based on distinguishing four fenofibrate experiments from four vehicle/control experiments was capable of distinguishing essentially all of the fenofibrate experiments from the non-fibrate compounds and controls, and further sorted most of the fibrate compounds accurately as well.

Individual drug signatures were obtained for each PPARα compound by deriving signatures discriminating between all treatments relating to an individual drug versus all other treatments. Thus, the individual drug signature highlights the differences in activity between members of the same class of therapeutic compound, and can identify potential side effects and/or possible synergies. For example, gemfibrozil administration induced 13 genes that were not induced by other PPARα agonists: 8 of the 13 genes are involved in cholesterol and fatty acid biosynthesis. This correlates with a known clinical contraindication. Fibrates are used to treat hyperlipoproteinemias, mainly by elevating the rate of fat oxidation in the liver, a mechanism corroborated by the up-regulation of the FABO pathway genes shown above. In many patients, particularly hypertriglyceridemic patients, gemfibrozil (but not other fibrates) induces an increase in LDL levels. Elevated fatty acid production raises VLDL and ILDL levels and subsequently LDL. The observation that gemfibrozil increases fatty acid/cholesterol biosynthesis gene expression may provide a molecular explanation of the paradoxical clinical effect.

A fenofibrate Drug Signature was constructed in order to test the ability of Drug Signatures to select individual compounds and experiments. The Drug Signature was calculated by comparing four fenofibrate experiments compared to four control/vehicle experiments, and was then used to sort 677 other experiments (where each combination of compound, dose, and time point constitutes an experiment). The sorted list was then graphed (FIG. 3), assigning a value of 1.0 to each fenofibrate experiment, a value of 0.5 to each fibrate other than fenofibrate, and a value of 0 to each non-fibrate control. The graph demonstrates that this minimal fenofibrate Drug Signature correctly sorts most fenofibrate experiments to the top of the list, most fibrate experiments near the top of the list (although lower than fenofibrate experiments), and all control experiments below the fenofibrate experiments (and below most of the fibrate experiments).

What is claimed:

1. A method for creating a Group Signature for a plurality of compounds having related activities, said method comprising:
a) providing a plurality of expression datasets, each expression dataset comprising the expression response of a first plurality of genes in a subject cell following exposure to a compound, wherein said plurality of expression datasets comprises an expression dataset for each of a plurality of test compounds having a similar or identical biological activity, and an expression dataset for each of a plurality of control compounds that lack the biological activity of the test compounds;
b) deriving a discrimination metric that distinguishes the plurality of test compounds from the control compounds based on gene expression to provide a distinctive gene set; and
c) selecting a second plurality of genes from said distinctive gene set to provide a Group Signature for said plurality of test compounds.

2. The method of claim 1, wherein step b) comprises:
i) ordering the expression datasets by Principal Component Analysis to provide a plurality of principal components;
ii) identifying the Principal Component that distinguishes the plurality of test compounds from the plurality of control compounds to the greatest degree, to provide a test Principal Component; and
iii) identifying the genes that distinguish the test Principal Component from the control compounds to the greatest degree to provide a distinctive gene set.

3. The method of claim 2, wherein said distinctive gene set is selected by identifying the genes that have the greatest eigenvalues in the test Principal Component.

4. The method of claim 1, wherein said discrimination metric comprises selecting a set of genes identified using the Golub distinction metric.

5. The method of claim 1, wherein said plurality of genes comprises at least 1,000 genes.

6. The method of claim 5, wherein said plurality of genes comprises at least 4,000 genes.

7. The method of claim 6, wherein said plurality of genes comprises at least 10,000 genes.

8. The method of claim 1, wherein the number of control compounds is less than the number of test compounds.

9. The method of claim 1, wherein said distinctive gene set comprises only upregulated genes.

10. The method of claim 2, wherein said distinctive gene set is selected by identifying the upregulated genes that have the greatest eigenvalues in the test Principal Component.

11. The method of claim 1, further comprising:
d) storing said expression datasets in a database; and
e) repeating steps a)-d) with a different set of test compounds.

12. The method of claim 1, further comprising:
d) contacting a subject cell expressing a plurality of proteins with each test compound; and
e) measuring the change in amount of each protein resulting from said contact to provide a protein response dataset for each compound.

13. The method of claim 12, further comprising:
f) storing said expression datasets and said protein response datasets in a database; and
g) repeating steps a)-f) with a different set of test compounds.

14. The method of claim 1, wherein said Group Signature consists of one to 50 genes.

15. The method of claim 14, wherein said Group Signature consists of one to 25 genes.

16. The method of claim 15, wherein said Group Signature consists of no more than three genes.

17. The method of claim 1, wherein said Group Signature comprises at least three genes.

18. The method of claim 17, wherein said Group Signature comprises at least 5 genes.

19. The method of claim 18, wherein said Group Signature comprises at least 10 genes.

20. The method of claim 19, wherein said Group Signature comprises at least 15 genes.

21. A method for creating a Group Signature for a plurality of compounds having related activities, said method comprising:
a) providing a plurality of test compounds having a similar or identical biological activity, and a plurality of control compounds that lack the biological activity of the test compounds;
b) contacting each compound with a subject cell;
c) measuring the expression response of a first plurality of genes for each subject cell to provide an expression dataset for each compound;
d) ordering the expression datasets by Principal Component Analysis to provide a plurality of principal components;
e) identifying the Principal Component that distinguishes the plurality of test compounds from the plurality of control compounds to the greatest degree, to provide a test Principal Component;
f) identifying the genes that distinguish the test Principal Component from the control compounds to the greatest degree to provide a distinctive gene set; and
g) selecting a second plurality of genes from said distinctive gene set to provide a Group Signature for said plurality of test compounds.

22. The method of claim 21, wherein said compounds are contacted with the cell in vivo.

23. A method for creating a Drug Signature capable of distinguishing the activity of a selected drug compound from a plurality of compounds having related activities, said method comprising:
a) providing a plurality of expression datasets, each expression dataset comprising the expression response of a plurality of genes in a subject cell following exposure to a compound, wherein said plurality of expression datasets comprises an expression dataset for said selected drug compound and an expression dataset for each of a plurality of test compounds having a similar or identical biological activity;
b) deriving a discrimination metric that distinguishes the selected drug compound from the plurality of test compounds based on gene expression to provide a distinctive gene set; and
c) selecting a plurality of genes from said distinctive gene set to provide a Drug Signature for said selected drug compound.

24. The method of claim 23, wherein step b) comprises:
i) ordering the expression datasets by Principal Component Analysis to provide a plurality of principal components;
ii) identifying the Principal Component that distinguishes the plurality of test compounds from the plurality of control compounds to the greatest degree, to provide a test Principal Component; and
iii) identifying the genes that distinguish the test Principal Component from the control compounds to the greatest degree to provide a distinctive gene set.

25. The method of claim 24, wherein said distinctive gene set is selected by identifying the genes that have the greatest eigenvalues in the test Principal Component.

26. The method of claim 23, wherein said discrimination metric comprises selecting a set of genes identified using the Golub distinction metric.

27. The method of claim 23, wherein said Drug Signature comprises at least three genes.

28. The method of claim 27, wherein said Drug Signature comprises at least five genes.

29. The method of claim 28, wherein said Drug Signature comprises at least ten genes.

30. The method of claim 23, wherein said Drug Signature consists of one to fifty genes.

31. The method of claim 30, wherein said Drug Signature consists of one to 25 genes.

32. The method of claim 31, where said Drug Signature consists of one to three genes.

33. The method of claim 23, wherein said Drug Signature comprises only upregulated genes.

34. A method for creating a Drug Signature capable of distinguishing the activity of a selected drug compound from a plurality of compounds having related activities, said method comprising:
a) providing said selected drug compound and a plurality of test compounds having a similar or identical primary biological activity;
b) contacting each compound with a subject cell;
c) measuring the expression response of a first plurality of genes for each subject cell to provide an expression dataset for each compound;
d) ordering the expression datasets by Principal Component Analysis to provide a plurality of principal components;
e) identifying the Principal Component that distinguishes the selected drug compound from said plurality of test compounds to the greatest degree, to provide a distinguishing Principal Component;
f) identifying the genes that contribute to the distinguishing Principal Component to the greatest degree to provide a distinguishing gene set; and
g) selecting a second plurality of genes from said distinguishing gene set to provide a Drug Signature for said selected drug compound.

35. The method of claim 34, wherein said compounds are contacted with the cell in vivo.

36. A Group Signature database, comprising:
a plurality of Group Signature records, wherein each Group Signature record comprises
indicia of at least one compound, wherein all compounds within a Group exhibit a similar or identical primary bioactivity;
indicia of a set of genes, wherein the expression of said genes is modulated in response to exposure to a compound having a primary bioactivity similar or identical to the primary bioactivity of a compound indicated in the Group record, and wherein said set of genes distinguishes said Group from all other Groups within said Group Signature database.

37. The Group Signature database of claim 36, wherein said plurality of Group Signature records comprises at least 10 Group Signature records.

38. The Group Signature database of claim 37, wherein said plurality of Group Signature records comprises at least 25 Group Signature records.

39. The Group Signature database of claim 36, wherein said set of genes for each Group Signature record comprises at least 5 genes.

40. The Group Signature database of claim 39, wherein said set of genes for each Group Signature record comprises at least 10 genes.

41. The Group Signature database of claim 36, wherein said set of genes for each Group Signature record consists of one to 50 genes.

42. The Group Signature database of claim 41, wherein said set of genes for each Group Signature record consists of one to 25 genes.

43. The Group Signature database of claim 36, wherein said database further comprises stress records, wherein each stress record comprises:
an indicia of a stress; and
indicia of a set of genes, wherein expression of said genes is modulated in response to said stress, and wherein said set of genes distinguishes said stress from all other stresses and Groups within said Group Signature database.

44. The Group Signature database of claim 43, wherein said stress is selected from the group consisting of elevated temperature, depressed temperature, elevated oxygen pressure, depressed oxygen pressure, elevated CO₂ pressure, depressed CO₂ pressure, starvation, dehydration, overcrowding, sleep deprivation, pain, infection, exposure to toxins, and light deprivation.

45. A Drug Signature database, comprising:
a plurality of Drug Signature records, wherein each Drug Signature record comprises
indicia of one compound; and
indicia of a set of genes, wherein expression of said genes is modulated in response to exposure to said compound, and wherein said set of genes distinguishes said compound from all other compounds within said Drug Signature database.

46. The Drug Signature database of claim 45, wherein said plurality of Drug Signature records comprises at least 10 records.

47. The Drug Signature database of claim 46, wherein said plurality of Drug Signature records comprises at least 50 records.

48. The Drug Signature database of claim 45, wherein said set of genes for each Drug Signature record comprises at least 5 genes.

49. The Drug Signature database of claim 48, wherein said set of genes for each Drug Signature record comprises at least 10 genes.

50. The Drug Signature database of claim 45, wherein said set of genes for each Drug Signature record consists of one to 50 genes.

51. The Drug Signature database of claim 50, wherein said set of genes for each Drug Signature record consists of one to 25 genes.

52. A method for determining the activity of a drug candidate, said method comprising:
a) providing a Group Signature database, said Group Signature database comprising a plurality of Group Signature records, wherein each Group Signature record comprises indicia of at least one compound, wherein all compounds within a Group exhibit a similar or identical primary bioactivity; and indicia of a set of genes, wherein expression of said genes is modulated in response to exposure to a compound having a primary bioactivity similar or identical to the primary bioactivity of a compound indicated in the Group record, and wherein said set of genes distinguishes said Group from all other Groups within said Group Signature database;
b) providing a drug candidate expression dataset for said drug candidate, said drug candidate expression dataset comprising the expression response of a plurality of genes in a subject cell following exposure to said drug candidate;
c) comparing said drug candidate expression dataset with each Group Signature;
d) selecting the Group Signature most similar to said drug candidate expression dataset;
e) identifying the activity of the drug candidate to be the primary bioactivity exhibited by the compounds within the most similar Group Signature.

53. The method of claim 52, wherein the similarity of the drug candidate expression dataset to each Group Signature is measured by a similarity score of S=Πₓ RelRkₓ.

54. The method of claim 52, wherein said drug candidate expression dataset consists of one to 200 genes.

55. The method of claim 54, wherein said Group Signature database further comprises bioassay data for each compound, and said drug candidate expression dataset further comprises bioassay data for said drug candidate.

56. A method for designing a Group Signature reagent, comprising:
a) providing a plurality of expression datasets, each expression dataset comprising the expression response of a first plurality of genes in a subject cell following exposure to a compound, wherein said plurality of expression datasets comprises an expression dataset for each of a plurality of test compounds having a similar or identical biological activity, and an expression dataset for each of a plurality of control compounds that lack the biological activity of the test compounds;
b) deriving a discrimination metric that distinguishes the plurality of test compounds from the control compounds based on gene expression to provide a distinctive gene set;
c) selecting a second plurality of genes from said distinctive gene set to provide a Group Signature for said plurality of test compounds; and
d) providing a set of polynucleotide probes capable of hybridizing specifically to one or more sequences of said second plurality of genes in said Group Signature to provide a Group Signature probe set.

57. The method of claim 56, wherein step b) comprises:
i) ordering the expression datasets by Principal Component Analysis to provide a plurality of principal components;
ii) identifying the Principal Component that distinguishes the plurality of test compounds from the plurality of control compounds to the greatest degree, to provide a test Principal Component; and
iii) identifying the genes that distinguish the test Principal Component from the control compounds to the greatest degree to provide a distinctive gene set.

58. The method of claim 57, wherein said distinctive gene set is selected by identifying the genes that have the greatest eigenvalues in the test Principal Component.

59. The method of claim 56, wherein said discrimination metric comprises selecting a set of genes identified using the Golub distinction metric.

60. The method of claim 56, further comprising:
e) repeating steps a)-d) to generate a plurality of different Group Signatures for unrelated compounds.

61. The method of claim 60, further comprising:
f) attaching said Group Signature probe set to a solid support in a defined location to form a Group Signature Array.

62. The method of claim 61, wherein said Group Signature Array comprises at least 100 Group Signature probe sets.

63. The method of claim 62, wherein said Group Signature Array comprises at least 500 Group Signature probe sets.

64. The method of claim 63, wherein said Group Signature Array comprises at least 1,000 Group Signature probe sets.

65. A Group Signature Array prepared in accordance with the method of claim 61.

66. A kit comprising a suitable container means, a Group Signature Array of claim 65, and instructions for using said kit.

67. A method for designing a Drug Signature reagent, comprising:
a) providing a plurality of expression datasets, each expression dataset comprising the expression response of a plurality of genes in a subject cell following exposure to a compound, wherein said plurality of expression datasets comprises an expression dataset for said selected drug compound and an expression dataset for each of a plurality of test compounds having a similar or identical biological activity;
b) deriving a discrimination metric that distinguishes the plurality of test compounds from the control compounds based on gene expression to provide a distinctive gene set;
c) selecting a plurality of genes from said distinguishing gene set to provide a Drug Signature for said selected drug compound; and
d) providing a set of polynucleotide probes capable of hybridizing specifically to the sequences of said genes in said Drug Signature to form a Drug Signature probe set.

68. The method of claim 67, wherein step b) comprises:
i) ordering the expression datasets by Principal Component Analysis to provide a plurality of principal components;
ii) identifying the Principal Component that distinguishes the plurality of test compounds from the plurality of control compounds to the greatest degree, to provide a test Principal Component; and
iii) identifying the genes that distinguish the test Principal Component from the control compounds to the greatest degree to provide a distinctive gene set.

69. The method of claim 68, wherein said distinctive gene set is selected by identifying the genes that have the greatest eigenvalues in the test Principal Component.

70. The method of claim 67, wherein said discrimination metric comprises selecting a set of genes identified using the Golub distinction metric.

71. The method of claim 67, further comprising:
e) repeating steps a)-d) to generate a plurality of different Drug Signatures for unrelated compounds.

72. The method of claim 67, further comprising:
e) attaching said Drug Signature probe set to a solid support in a defined location to form a Drug Signature Array.

73. The method of claim 67, wherein said Drug Signature Array comprises at least 100 Drug Signature probe sets.

74. The method of claim 73, wherein said Drug Signature Array comprises at least 500 Drug Signature probe sets.

75. The method of claim 74, wherein said Drug Signature Array comprises at least 1,000 Drug Signature probe sets.

76. The method of claim 75, wherein said Drug Signature Array comprises at least 10,000 Drug Signature probe sets.

77. A Drug Signature Array prepared in accordance with the method of claim 72.

78. A kit comprising a suitable container means, a Drug Signature Array of claim 77, and instructions for using said kit.

79. A method for determining the activity of a drug candidate, said method comprising:
a) providing a Group Signature Array, said Group Signature Array comprising a solid support having affixed thereto a plurality of Group Signature probe sets, wherein each Group Signature probe set comprises a set of polynucleotide probes capable of hybridizing specifically to the sequences of the genes in each Group Signature, wherein said Group Signatures are obtained by:
i) providing a plurality of expression datasets, each expression dataset comprising the expression response of a plurality of genes in a subject cell following exposure to a compound, wherein said plurality of expression datasets comprises an expression dataset for each of a plurality of test compounds having a similar or identical biological activity, and an expression dataset for each of a plurality of control compounds that lack the biological activity of the test compounds;
ii) deriving a discrimination metric that distinguishes the plurality of test compounds from the control compounds based on gene expression to provide a distinctive gene set;
iii) selecting a plurality of genes from said distinctive gene set to provide a Group Signature for said plurality of test compounds; and
iv) repeating steps i)-iii) for each Group Signature;
b) contacting a subject cell with said drug candidate;
c) extracting mRNA from said subject cell;
d) reverse-transcribing said mRNA to cDNA;
e) contacting said Group Signature Array with said cDNA; and
f) determining whether any Group Signature probe set exhibits increased binding of cDNA.

80. A method for screening a library of compounds, wherein the library comprises a plurality of drug candidates, comprising:
a) determining the activity of each drug candidate according to the method of claim 79; and
b) selecting a drug candidate, wherein the Group Signature probe set exhibits increased binding to the cDNA that results from contacting the subject cell with said drug candidate.

81. A polynucleotide probe set for detecting fibrate-like activity, the set comprising:
a plurality of polynucleotides capable of hybridizing specifically to genes selected from the group consisting of Rat for cytochrome P452, Rat cytochrome P450, Rat cytochrome P450-LA-omega (lauric acid omega-hydroxylase), Rat Sulfotransferase K2, Rat cytochrome P450-LA-omega (lauric acid omega-hydroxylase), Rat Cyp4a locus, encoding cytochrome P450 (IVA3), Rat cytochrome P450, Rat mitochondrial 3-2-trans-enoyl-CoA isomerase, Rat carnitine octanoyltransferase, Rat Wistar peroxisomal enoyl hydratase-like protein (PXEL), Rat mitochondrial long-chain 3-ketoacyl-CoA thiolase β-subunit of mitochondrial trifunctional protein, Rat liver fatty acid binding protein (FABP), Rat pyruvate dehydrogenase kinase isoenzyme 4 (PDK4), Rat mitochondrial isoform of cytochrome b5, Hypothetical protein Rv3224, Rat peroxisomal enoyl-CoA: hydrotase-3-hydroxyacyl-CoA bifunctional enzyme, Rat peroxisomal membrane protein Pmp26p (Peroxin-11), Rat acyl-CoA hydrolase, Rat acyl-CoA oxidase, Rat acyl-CoA hydrolase, Rat 2,4-dienoyl-CoA reductase precursor, Rat mitochondrial 3-hydroxy-3-methylglutaryl-CoA synthase, Rat peroxisomal enoyl-CoA: hydrotase-3-hydroxyacyl-CoA bifunctional enzyme, and Mouse peroxisomal long chain acyl-CoA thioesterase Ib (Pte1b).

82. The polynucleotide probe set of claim 81, wherein said plurality of polynucleotides are capable of hybridizing specifically to at least 3 genes.

83. The polynucleotide probe set of claim 82, wherein said plurality of polynucleotides are capable of hybridizing specifically to at least 5 genes.

84. The polynucleotide probe set of claim 83, wherein said plurality of polynucleotides are capable of hybridizing specifically to at least 10 genes.

85. A kit comprising a suitable container means, a polynucleotide probe set of claim 81, and instructions for using said kit.

86. A polynucleotide probe set for detecting gemfibrozil-like activity, the set comprising:
a plurality of polynucleotides capable of hybridizing specifically to genes selected from the group consisting of Rat fatty acid synthase, Rat cholesterol 7α-hydroxylase, Mouse acetyl-CoA synthetase, Mouse Vanin-1, Rat kidney-specific protein (KS), Rat 2,3-oxidosqualene:lanosterol cyclase, Rat aldehyde dehydrogenase, and Rat thymosin β-10.

87. The polynucleotide probe set of claim 86 wherein said plurality of polynucleotides are capable of hybridizing specifically to at least 3 genes.

88. The polynucleotide probe set of claim 87 wherein said plurality of polynucleotides are capable of hybridizing specifically to at least 5 genes.

89. The polynucleotide probe set of claim 88 wherein said plurality of polynucleotides are capable of hybridizing specifically to at least 10 genes.

90. A kit comprising a suitable container means, a polynucleotide probe set of claim 86, and instructions for using said kit.

91. A method for screening drug candidates for fibrate activity, the method comprising:
a) contacting a subject cell with a drug candidate;
b) extracting mRNA from said subject cell;
c) reverse-transcribing said mRNA into cDNA;
d) hybridizing said cDNA to a fibrate signature probe set, said probe set comprising a plurality of polynucleotides capable of hybridizing specifically to a fibrate signature gene, wherein said fibrate signature genes are selected from the group consisting of Rat for cytochrome P452, Rat cytochrome P450, Rat cytochrome P450-LA-omega (lauric acid omega-hydroxylase), Rat Sulfotransferase K2, Rat cytochrome P450-LA-omega (lauric acid omega-hydroxylase), Rat Cyp4a locus, encoding cytochrome P450 (IVA3), Rat cytochrome P450, Rat mitochondrial 3-2-trans-enoyl-CoA isomerase, Rat carnitine octanoyltransferase, Rat Wistar peroxisomal enoyl hydratase-like protein (PXEL), Rat mitochondrial long-chain 3-ketoacyl-CoA thiolase β-subunit of mitochondrial trifunctional protein, Rat liver fatty acid binding protein (FABP), Rat pyruvate dehydrogenase kinase isoenzyme 4 (PDK4), Rat mitochondrial isoform of cytochrome b5, Hypothetical protein Rv3224, Rat peroxisomal enoyl-CoA: hydrotase-3-hydroxyacyl-CoA bifunctional enzyme, Rat peroxisomal membrane protein Pmp26p (Peroxin-11), Rat acyl-CoA hydrolase, Rat acyl-CoA oxidase, Rat acyl-CoA hydrolase, Rat 2,4-dienoyl-CoA reductase precursor, Rat mitochondrial 3-hydroxy-3-methylglutaryl-CoA synthase, Rat peroxisomal enoyl-CoA: hydrotase-3-hydroxyacyl-CoA bifunctional enzyme, and Mouse peroxisomal long chain acyl-CoA thioesterase Ib (Pte1b); and
e) determining if said subject cell exhibits increased expression of a fibrate signature gene.

92. A database product, comprising:
a computer-readable medium, said medium storing thereon a Group Signature database, said database comprising a plurality of Group Signature records, wherein each Group Signature record comprises indicia of at least one compound, wherein all compounds within a Group exhibit a similar or identical primary bioactivity; and indicia of a set of genes, wherein expression of said gene is modulated in response to exposure to a compound having a primary bioactivity similar or identical to the primary bioactivity of a compound indicated in the Group record, and wherein said set of genes distinguishes said Group from all other Groups within said Group Signature database.
